(19) United States
(12) Patent Application Publication
(10) Pub. No.: US 2025/0009730 A1
(43) Pub. Date: Jan. 9, 2025

(54) IFENPRODIL SALTS AND THERAPEUTIC USES THEREOF

(71) Applicant: Algernon Pharmaceuticals Inc., Vancouver (CA)

(72) Inventor: Christopher BRYAN, Vancouver (CA)

(21) Appl. No.: 18/704,395

(22) PCT Filed: Oct. 24, 2022
(86) PCT No.: PCT/CA2022/051566

§ 371 Date: Apr. 24, 2024

Related U.S. Application Data
(60) Provisional application No. 63/271,471, filed on Oct. 25, 2021.
(60) Provisional application No. 63/271,476, filed on Oct. 25, 2021.
(60) Provisional application No. 63/271,484, filed on Oct. 25, 2021.
(60) Provisional application No. 63/271,494, filed on Oct. 25, 2021.
(60) Provisional application No. 63/271,503, filed on Oct. 25, 2021.

(51) Int. Cl. A61K 31/445; A61K 45/06; C07D 211/14

(52) U.S. Cl. CPC A61K 31/445; A61K 45/06; C07D 211/14

(57) ABSTRACT

The salts ifenprodil oleate, ifenprodil nicotinate, ifenprodil pamoate, ifenprodil fumarate, and ifenprodil benzoate are prepared and found useful in the treatment and/or prevention of a myriad of diseases and conditions.

### CROSS REFERENCE TO RELATED APPLICATIONS

The present application claims priority to U.S. provisional application No. 63/271,471, filed Oct. 25, 2021, U.S. provisional application No. 63/271,476, filed Oct. 25, 2021, U.S. provisional application No. 63/271,484, filed Oct. 25, 2021, U.S. provisional application No. 63/271,494, filed Oct. 25, 2021, and U.S. provisional application No. 63/271,503, filed Oct. 25, 2021.

### FIELD OF THE INVENTION

The present invention is related to compounds having NR2B modulating properties, pharmaceutical compositions comprising these compounds, and their use in the treatment of diseases associated with NR2B receptor activity in animals, in particular humans.

### BACKGROUND OF THE INVENTION

Glutamate is one of the major excitatory neurotransmitters that is widely spread in the brain. The first indications of its role as an excitatory messenger was in the 1950's when it was observed that intravenous administration of glutamate induces convulsions. However, the detection of the whole glutamatergic neurotransmitter system with its various receptors did not take place before the 1970's and 1980's when numerous antagonists were developed or, as in the case of PCP and ketamine, were identified as antagonists. Finally, in the 1990's molecular biology provided the tools for the classification of the glutamatergic receptors.

N-methyl-D-aspartate (NMDA) receptors are a subtype of ionotropic glutamate receptors that mediate excitatory synaptic transmission in the brain. NMDA receptors are ubiquitously distributed throughout the brain and play a key role in synaptic plasticity, synaptogenesis, excitotoxicity, memory acquisition and learning. NMDA receptors are distinct from other major subtypes of ionotropic glutamate receptors (AMPA and kainate receptors) in that they are blocked by Mg²⁺ at resting membrane potentials, are highly Ca²⁺ permeable, and require co-activation by two distinct neurotransmitters: glutamate and glycine (or D-serine) (Traynelis S F et al., Pharmacol Rev. 2010; 62(3):405-96). The influx of Ca²⁺ through NMDA receptors triggers signaling cascades and regulates gene expression that is critical for different forms of synaptic plasticity including both long-term potentiation of synapse efficacy (LTP) (Berberich S et al., Neuropharmacology 2007; 52(1):77-86) and long-term depression (LTD) (Massey, P V et al., J Neurosci. 2004 Sep. 8; 24(36):7821-8).

The vast majority of the mammalian NMDA receptors form a heterotetramer made of two obligatory GluN1 (also called NR1) units and two variable GluN2 (also called NR2) receptor subunits, encoded by the GRIN1 gene and one of four GRIN2 genes, respectively. The variable subunits are selected from one or more of the four GluN2subunits: GluN2A (NR2A), GluN2B (NR2B), GluN2C (NR2C), and GluN2D (NR2D).

Different NR2 subunits confer distinct electrophysiological and pharmacological properties on the receptors and couple them with different signaling machineries. For instance, it has been suggested that NR2A- and NR2B-containing NMDA receptor subtypes have opposing roles in dictating the direction of synaptic plasticity (Kirson et al., 1996; Tovar et al., 1999; Sheng et al., 1994; Liu et al., 2004). It has been demonstrated using heteromeric NMDA receptors expressed in Xenopus oocytes that oocyte-expressed NR1/NR2A receptors display a higher affinity for certain antagonists and a slightly lower affinity for selected agonists than NR1/NR2B receptors (Buller et al., 1994). The distribution of NR2A mRNA has been correlated with the distribution of “antagonist-preferring” NMDA receptors, defined by high-affinity 3H-2-carboxypiperazine-4-yl-propyl-1-phosphonic (3H-CPP) binding sites. Accordingly, there is evidence that NMDA receptor antagonists may preferentially target NR2A-containing NMDA receptors. NR2B subunits are expressed primarily in the forebrain in the adult mammalian brain (Paoletti P et al., Nat Rev Neurosci. 2013; 14(6):383-400; Watanabe Metal., J Comp Neurol. 1993; 338(3):377-90) and are implicated in learning, memory processing, mood, attention, emotion and pain perception (Cull-Candy S et al., Curr Opin Neurobiol. 2001; 11(3):327-35).

Activation of the NMDA receptor may induce programmed cell death (apoptosis) in neurons, and may underlie the loss of neurons and neuronal function in central nervous system disorders ranging from acute brain trauma and stroke to neurodegenerative diseases such as Huntington's, Alzheimer's, and Parkinson's Diseases (Mattson, 2000; Graham et al., 2001; Yu et al., 2001; Nicotera et al., 1999; Hardingham et al., 2002).

Neuronal apoptosis induced by activation of the NMDA receptor is thought to be central to the loss of neurons and neuronal function that accompanies stroke, brain trauma and neurodegenerative disorders. The effect of NMDA receptor antagonism illustrates two apparently paradoxical roles: both neuronal apoptosis in developmental models and neuroprotection against ischemic brain damage in stroke models (Hardingham et al., 2002; Ikonomidou et al., 1999; Lee et al., 1999; Arundine et al., 2004). A variety of NMDA antagonists, such as ifenprodil and eliprodil, are thought to have neuroprotective effects. Ro 63-1908, an NMDA ligand having 20,000-fold selectivity for the NR1C and NR2B receptors over NR1C+NR2A receptors, reportedly has a dose-related neuroprotective effect against cortical damage in a model of permanent focal ischemia (Gill et al., 2002).

There has been a significant degree of interest in the clinical relevance of NR2B selective antagonists (McCauley, 2005; WO2005080317). NR2B selective antagonists (such as CP-101,606; CI-1041; Co-101,244, RG-13579 and RG-1103) have shown promise in some neuroprotective treatments (Nagy et al., 2004). A significant number of NR2B-selective antagonists have been identified (Donevan et al., 2000; White et al, 2000). For example, felbamate, an anticonvulsant used in the treatment of seizures, has been characterized as an NR2B-selective antagonist (Kleckner et al., 1999). A wide variety of NR2B-containing NMDA receptor antagonists have reportedly been the subject of clinical testing, for a wide variety of indications: EVT-101, EVT-103 and EVT-102 (Evotec) for Alzheimer's and Parkinson's diseases and neuropathic pain; RGH-896 (Gedeon Richter) for neuropathic pain and other CNS indications; ED-1529 (Sosei) for neuropathic pain and other pain indications; HON-0001 (Taisho) for neuropathic and other pain conditions; Traxoprodil mesylate (Pfizer) for analgesia and stroke; Ifenprodil (Sanofi) for peripheral neuropathies and CNS neurodegenerative disorders (EP698391).

NMDA receptor glycine agonists have also been the subject of extensive clinical testing: Nebostinel (Rottapharm) as an antidepressive, antipsychotic, and for cognition disorders (AD, depression, schizophrenia), and age-associated memory impairment; NT-13 (Nyxis Neurotherapies) for neuropathic pain, prevention of stroke and for cognition enhancement; SC-49088 (Pfizer) for Alzheimer's disease and age-associated memory impairment.

Compounds that modulate NR2B-containing NMDA receptor function can be useful in prevention and/or treatment of many neurological and psychiatric disorders including but not limited to bipolar disorder (Martucci L et al., Schizophrenia Res, 2006; 84(2-3):214-21) major depressive disorder (Miller O H et al., eLife. 2014; 3:e03581; Li N et al., Biol Psychiatry. 2011; 69(8):754-61; Peng W F, et al. Epilepsy Res. 2016 January; 119:77-85; Yao Y, et al. Psychopharmacology (Berl). 2020 May; 237 (5): 1421-1433; Poleszak E, et al. Prog Neuropsychopharmacol Biol Psychiatry. 2013 Oct. 1;46:29-35; Li S X, et al. Mol Psychiatry. 2018 Mar;23(3):597-608), treatment-resistant depression (Preskorn S H et al. J Clin Psychopharmacol. 2008; 28(6):631-7) and mood disorders (including schizophrenia (Grimwood S et al., Neuroreport. 1999; 10(3):461-5; Weickert C S et al. Molecular Psychiatry (2013) 18, 1185-1192), ante-and postpartum depression, seasonal affective disorder and the like), Alzheimer's disease (Hanson J E et al., Neurobiol Dis. 2015; 74:254-62; Li S et al., J Neurosci. 2011; 31(18):6627-38; Hu N W, et al. Proc Natl Acad Sci U S A. 2009 Dec. 1; 106(48):20504-9) and other dementias (Orgogozo J M et al. Stroke 2002, 33:1834-1839; Xu C S, et al. J Neurochem. 2015 Aug; 134(3):566-77), Parkinson's disease (Duty S, CNS Drugs. 2012; 26(12):1017-32, Steece-Collier K et al., Exp Neurol. 2000; 163(1):239-43; Leaver K R et al. Clin Exp Pharmacol Physiol. 2008; 35(11):1388-94; Nash J E, et al. Exp Neurol. 2000 Sep; 165(1):136-42; Xinyu Zhao, et al. bioRxiv 2021.07.28.454206), Huntington's chorea (Tang T S et al., Proc Natl Aced Sci USA. 2005; 102(7):2602-7; Li L et al., J Neurophysiol. 2004; 92(5):2738-46), multiple sclerosis (Grasselli G et al., Br J Pharmacol. 2013; 168(2):502-17;Farjam M et al., Iran J Pharm Res. 2014; 13(2):695-705), such as cognitive impairment (Wang D et al. 2014, Expert Opin Ther Targets Expert Opin Ther Targets. 2014; 18(10):1121-30), head injury (Bullock M R et al., Ann N Y Acad Sci. 1999; 890:51-8), spinal cord injury, stroke (Yang Y et al., J Neurosurg. 2003; 98(2):397-403; Zhang Z, et al. Exp Neurol. 2018 Mar; 301 (Pt A):13-25; Picconi B, et al. Stroke. 2006 Jul; 37(7):1895-901; Sun J Y, et al. Transl Stroke Res. 2021 Mar. 12), epilepsy (Naspolini A P et al., Epilepsy Res. 2012 June; 100(1-2):12-9), movement disorders (e.g. dyskinesias) (Morissette M et al., Mov Disord. 2006; 21(1):9-17), various neurodegenerative diseases (e.g. amyotrophic lateral sclerosis (Fuller P I et al., Neurosci Lett. 2006; 399(1-2):157-61) or neurodegeneration associated with bacterial or chronic infections, glaucoma (Naskar R et al. Semin Ophthalmol. 1999Sep. 14(3):152-8), pain (e.g. chronic, cancer, post-operative and neuropathic pain (Wu L J and Zhuo M, Neurotherapeutics. 2009; 6(4):693-702), diabetic neuropathy, migraine (Peeters M et al., J Pharmacol Exp Ther. 2007; 321(2):564-72), cerebral ischemia (Yuan H et al., Neuron. 2015; 85(6):1305-18), encephalitis (Dalmau J. et al., Lancet Neurol. 2008; 7(12):1091-8.), autism and autism spectrum disorders (Won H. et al., Nature. 2012; 486(7402):261-5), memory and learning disorders (Tang, Y. P. et al., Nature. 1999; 401(6748):63-9) such as Korsakoffs disease, obsessive compulsive disorder (Arnold P D et al., Psychiatry Res. 2009; 172(2):136-9.), attention deficit hyperactivity disorder (ADHD) (Dorval K M et al., Genes Brain Behay. 2007; 6(5):444-52), PTSD (Haller J et al. Behav Pharmacol. 2011; 22(2):113-21; Leaderbrand K et al. Neurobiol Learn Mem. 2014; 113:35-40; Sasaki T, et al. Psychother Psychosom 2013;82:344-345.), tinnitus (Guitton M J, and Dudai Y, Neural Plast. 2007; 80904; Hu S S et al. 2016; 273(2):325-332), sleep disorders (like narcolepsy or excessive daytime sleepiness, patent WO 2009058261 A1), vertigo and nystagmus (Straube A. et al., Curr Opin Neurol. 2005, 18(1):11-4, Starck M et al. J Neurol. 1997 January, 244(1):9-16), anxiety autoimmunological disorders like neuropsychiatric systemic lupus erythematosus (Kowal C et al. Proc. Natl. Acad. Sci. U.S.A. 2006; 103, 19854-19859) and addictive illnesses (e.g. alcohol addiction, drug addiction) (Sugaya N, et al. Neuropsychopharmacol Rep. 2018 Mar;38(1):9-17; Nagy J, 2004, Curr Drug Targets CNS Neurol Disord. 2004; 3(3):169-79.; Shen H et al., Proc Natl Aced Sci USA. 2011; 108(48):19407-12; Szumlinski K K, et al. J Drug Abuse. 2016;2(2):22; Schilström B, et al. J Neurosci. 2006 Aug. 16;26(33):8549-58; Bingor A, et al. J Mol Neurosci. 2021 Jan. 21; Schumann J, et al. Neurosci Lett. 2009 Sep. 18;461(2):159-62; Witkin, J. M. et al. Behavioural Pharmacology, 6(3), 245-253; Haowei Shen, et al. Proceedings of the National Academy of Sciences November 2011, 108 (48) 19407-19412; Li L, et al. Neurochem Res. 2016 Oct;41(10):2636-2644; Chen G, et al. Neurochem Res. 2020 April;45(4):891-901; Ma Y Y, et al. Neurochem Res. 2011 Mar; 36(3):383-91; Suzuki T, et al. Life Sci. 1999;64(12):PL151-6; Liu X S, et al. CNS Neurosci Ther. 2014 Sep;20(9):823-9; Ma YY, et al. Exp Neurol. 2006 Aug; 200(2):343-55), acute lung injury (PCT/CA2020/050199); liver disease related neurologic alterations (Taoro-Gonzalez et al. Journal of Neuroinflammation (2018) 15:36 https://doi.org/10.1186/s12974-018-1082-z); cerebral palsy (Romain H. et al, Proceedings of the National Academy of Sciences October 2008, 105 (43) 16779-16784; DOI:10.1073/pnas.0803004105); neuronal reperfusion injury (Doğan A, et al, J Neurosurg. 1997 Dec; 87(6):921-6; Kikuchi, T. et al, Journal of Pharmaceutical Sciences 108(12):3823-3830); neuronal hemorrhage (Sun J Y, et al, Transl Stroke Res. 2021 Mar. 12. doi: 10.1007/s12975-021-00906-4. Epub ahead of print. PMID: 33713028; Zhang Z, et al., Exp Neurol. 2018 Mar;301 (Pt A):13-25. doi: 10.1016/j.expneurol.2017.12.005. Epub 2017 Dec. 16. PMID: 29258835; MI Qiong-jie, et al, Chin J Pathophysiolo, 2019, 35(8): 1387-1392.); and neuronal exposure to a toxic substance (Xinyu Zhao, et al, bioRxiv 2021.07.28.454206; doi https://doi.org/10.1101/2021.07.28.454206; Hrnčić D, et al., Pharmacology 2009;84:234-239. doi: 10.1159/000238055).

Compounds that modulate NR2B-containing NMDA receptor function can be useful in treatment and/or prevention of other disorders or conditions, including elevated cholesterol (Kim H Y, et al. Chem Res Toxicol. 2016 May 16;29(5):892-900), idiopathic pulmonary fibrosis (PCT Application No. PCT/CA2020/050199), chronic cough (PCT Application No. PCT/CA2020/050306), and viral infections (Zhang C, et al. mSystems. 2019 Dec. 10;4(6):e00431-19; Xiao J, et al. Immunity. 2020 Jan. 14;52(1):109-122; Kim H Y, et al. Chem Res Toxicol. 2016 May 16;29(5):892-900).

As used in the prior art, “Ifenprodil” almost always refers to Ifenprodil hemitartrate. The utility and efficacy of other Ifenprodil salts was unknown until now.

In view of the clinical importance of NR2B, there is a need for the identification of compounds that modulate NR2B receptor function for the development of new therapeutic agents. Such compounds are provided herein.

### SUMMARY OF THE INVENTION

One aspect of this invention concerns the compound Ifenprodil oleate having the Formula:

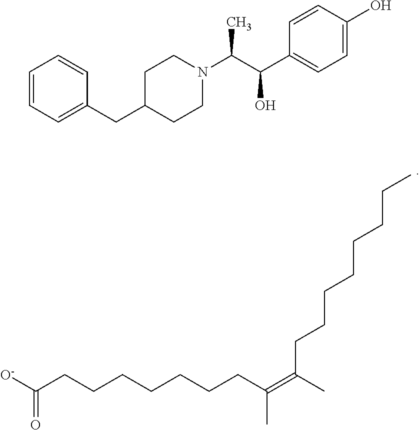 [SMILES: C[C@@H]([C@H](O)c1ccc(O)cc1)N1CCC(Cc2ccccc2)CC1 ; CCCCCCCC/C(C)=C(/C)CCCCCCCC(=O)[O-]]

Another aspect of this invention concerns the compound Ifenprodil nicotinate having the Formula:

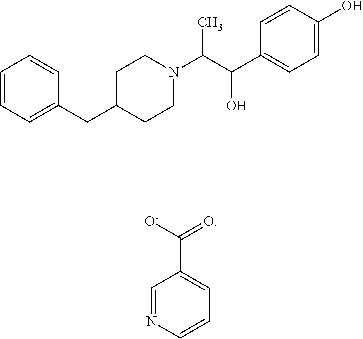 [SMILES: CC(C(O)c1ccc(O)cc1)N1CCC(Cc2ccccc2)CC1 ; O=C([O-])c1cccnc1]

Yet another aspect of this invention concerns the compound Ifenprodil pamoate having the Formula:

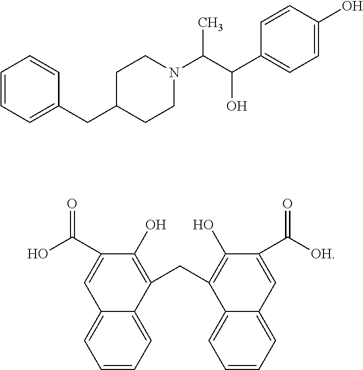 [SMILES: CC(C(O)c1ccc(O)cc1)N1CCC(Cc2ccccc2)CC1 ; O=C(O)c1cc2ccccc2c(Cc2c(O)c(C(=O)O)cc3ccccc23)c1O]

A further aspect of this invention concerns the compound Ifenprodil fumarate having the Formula:

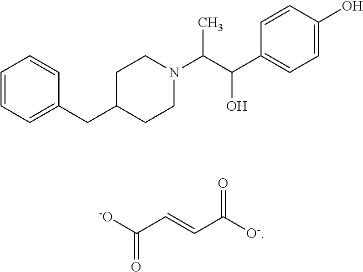

A still further aspect of this invention concerns the compound Ifenprodil benzoate having the Formula:

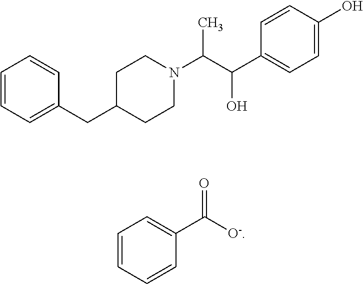

Thus the invention teaches a compound comprising an ifenprodil salt selected from the group consisting of ifenprodil oleate, ifenprodil nicotinate, ifenprodil pamoate, ifenprodil fumarate, and ifenprodil benzoate.

The invention also teaches a method of treating or preventing a disorder or condition comprising administering an ifenprodil salt selected from the group consisting of ifenprodil oleate, ifenprodil nicotinate, ifenprodil pamoate, ifenprodil fumarate, and ifenprodil benzoate to a subject in need thereof.

The invention further teaches the use of an ifendprodil salt selected from the group consisting of ifenprodil oleate, ifenprodil nicotinate, ifenprodil pamoate, ifenprodil fumarate, and ifenprodil benzoate for the treatment or prevention of a disorder or condition in a subject in need thereof.

Thus the invention teaches a compound comprising an ifenprodil salt selected from the group consisting of ifenprodil oleate, ifenprodil nicotinate, ifenprodil pamoate, ifenprodil fumarate, and ifenprodil benzoate for the treatment or prevention of a disorder or condition in a subject in need thereof.

The methods, uses, or compounds of the invention can be used for mood affective disorders; neurotic, stress-related and somatoform disorders including anxiety disorders; disorders of psychological development; behavioral syndromes associated with physiological disturbances and physical factors; extrapyramidal and movement disorders; episodic and paroxysmal disorders, epilepsy; pain; forms of neurodegeneration; cerebrovascular diseases, acute and chronic; and any sequelae of cerebrovascular diseases.

The methods, uses, or compounds of the invention can be used for bipolar disorder, major depressive disorder, treatment-resistant depression, schizophrenia, ante-and postpartum depression, seasonal affective disorder, Alzheimer's disease, dementia, Parkinson's disease, Huntington's chorea, multiple sclerosis, cognitive impairment, head injury, spinal cord injury, stroke, epilepsy, movement disorders including dyskinesia, neurodegenerative diseases including amyotrophic lateral sclerosis and neurodegeneration associated with bacterial or chronic infections, glaucoma, pain including chronic, cancer, post-operative and neuropathic pain, diabetic neuropathy, migraine, cerebral ischemia, encephalitis, autism and autism spectrum disorders, memory and learning disorders, obsessive compulsive disorder, attention deficit hyperactivity disorder (ADHD), PTSD, tinnitus, sleep disorders including narcolepsy and excessive daytime sleepiness, vertigo and nystagmus, anxiety autoimmunological disorders including neuropsychiatric systemic lupus erythematosus, addictive illnesses including alcohol addiction and drug addiction, viral infections, acute lung injury, liver disease related neurological alterations, Korsakoff's disease, cerebral palsy, neuronal reperfusion injury, neuronal hemorrhage, tinnitus, neuronal exposure to a toxic substance, elevated cholesterol, idiopathic pulmonary fibrosis, fibrotic injury, radiation induced fibrosis, and/or chronic cough.

The methods, uses, or compounds of the invention can further comprise administration with a compound that modulates NR2A-containing NMDA receptors.

### BRIEF DESCRIPTION OF THE DRAWINGS

The present invention will now be described in more detail having regard to the drawings in which:

FIG. 1 shows numeric data and plot representation of agonist effects of test articles, vehicle control, and positive controls antagonists. All values were normalized relatively to current elicited with 100 μM glutamate and 50 μM glycine after second application of glutamate. Peak current amplitude measurements are shown;

FIG. 2 shows numeric data and plot representation of antagonist effects of test articles and positive controls antagonists co-applied with 2 μM glutamate and 50 μM glycine. All values were normalized relatively to current elicited with 100 μM glutamate and 50 μM glycine. Peak current amplitude measurements are shown.

FIG. 3 shows numeric data and plot representation of agonist effects of test articles, vehicle control, and positive control antagonists. All values were normalized relatively to current elicited with 100 μM glutamate and 50 μM glycine during second application of glutamate. Steady state current amplitude measurements are shown;

FIG. 4 shows numeric data and plot representation of antagonist effects of test articles and positive controls antagonists co-applied with 2 μM glutamate and 50 μM glycine. All values were normalized relatively to current elicited with 100 μM glutamate and 50 μM glycine. Steady state current amplitude measurements are shown (4-6 seconds after application of glutamate);

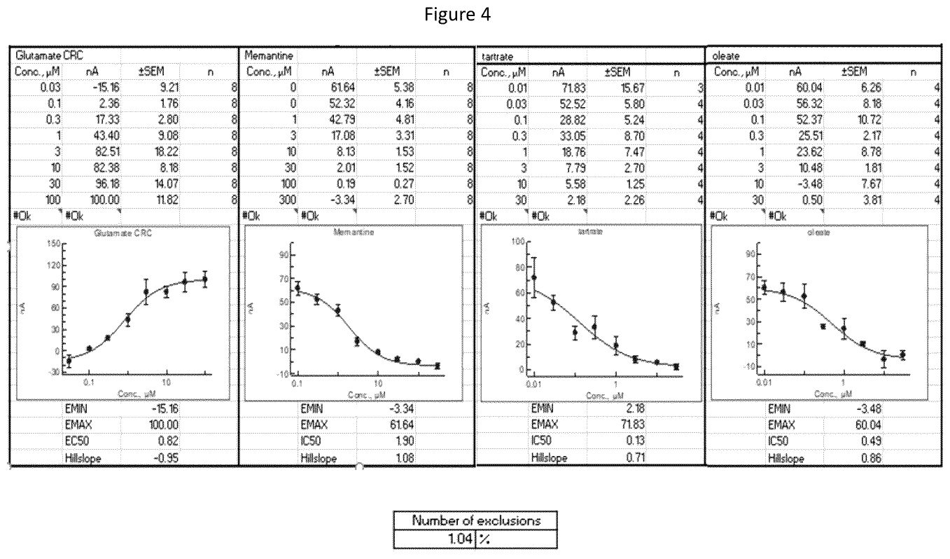

FIG. 5 is an NMR data plot of the ifenprodil oleate;

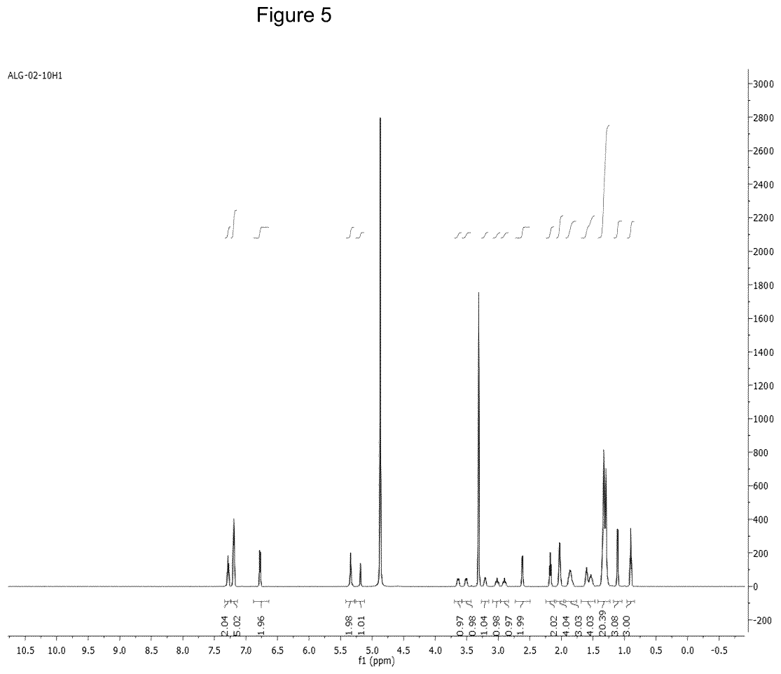

FIG. 6 shows numeric data and plot representation of agonist effects of test articles, vehicle control, and positive controls antagonists. All values were normalized relatively to current elicited with 100 μM glutamate and 50 μM glycine after second application of glutamate. Peak current amplitude measurements are shown;

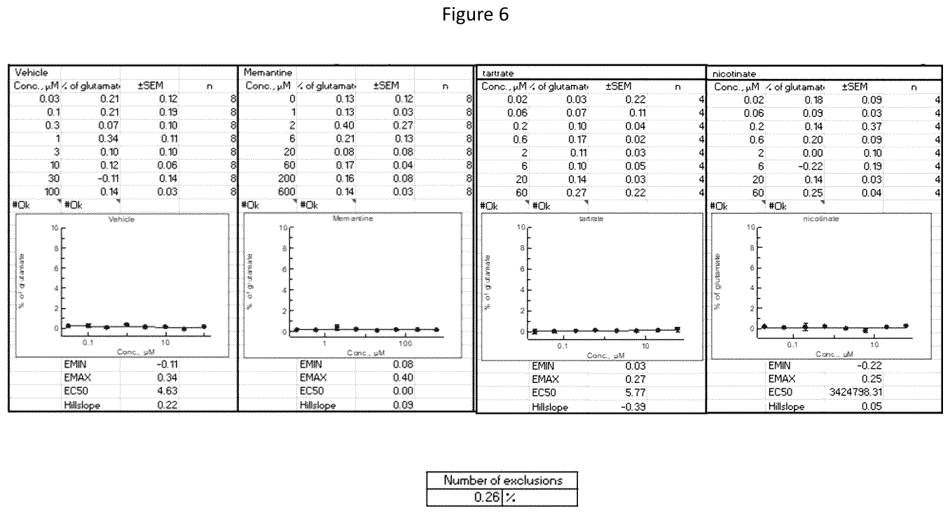

FIG. 7 shows numeric data and plot representation of antagonist effects of test articles and positive controls antagonists co-applied with 2 μM glutamate and 50 μM glycine. All values were normalized relatively to current elicited with 100 μM glutamate and 50 μM glycine. Peak current amplitude measurements are shown.

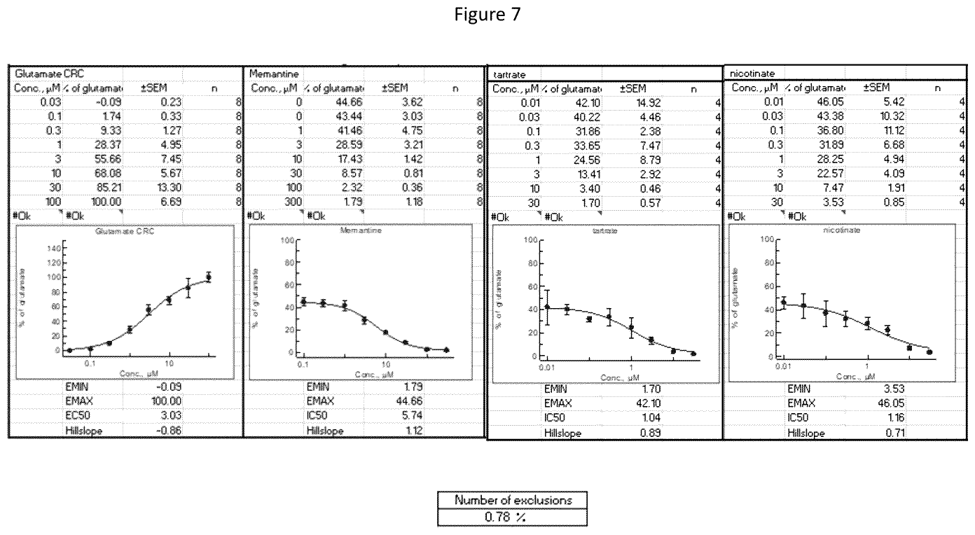

FIG. 8 shows numeric data and plot representation of agonist effects of test articles, vehicle control, and positive control antagonists. All values were normalized relatively to current elicited with 100 μM glutamate and 50 μM glycine during second application of glutamate. Steady state current amplitude measurements are shown;

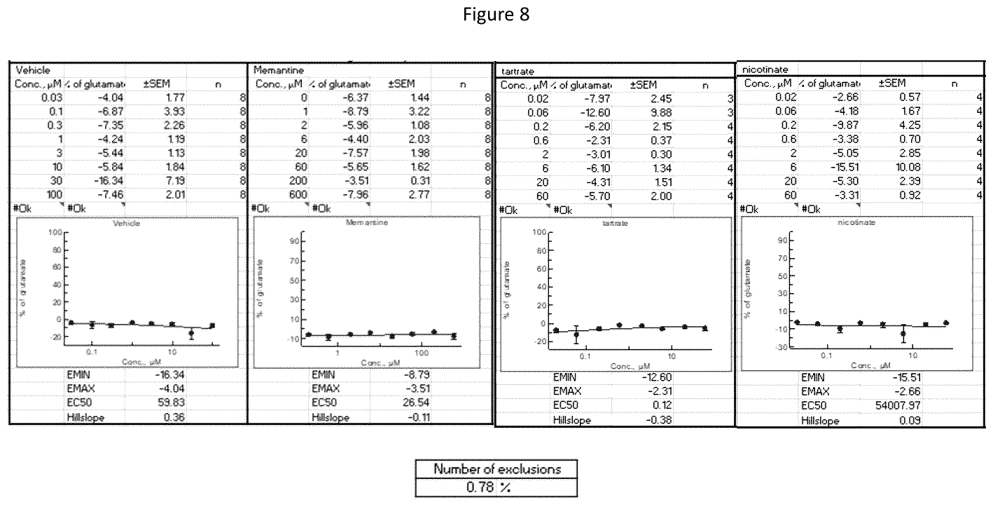

FIG. 9 shows numeric data and plot representation of antagonist effects of test articles and positive controls antagonists co-applied with 2 μM glutamate and 50 μM glycine. All values were normalized relatively to current elicited with 100 μM glutamate and 50 μM glycine. Steady state current amplitude measurements are shown (4-6 seconds after application of glutamate);

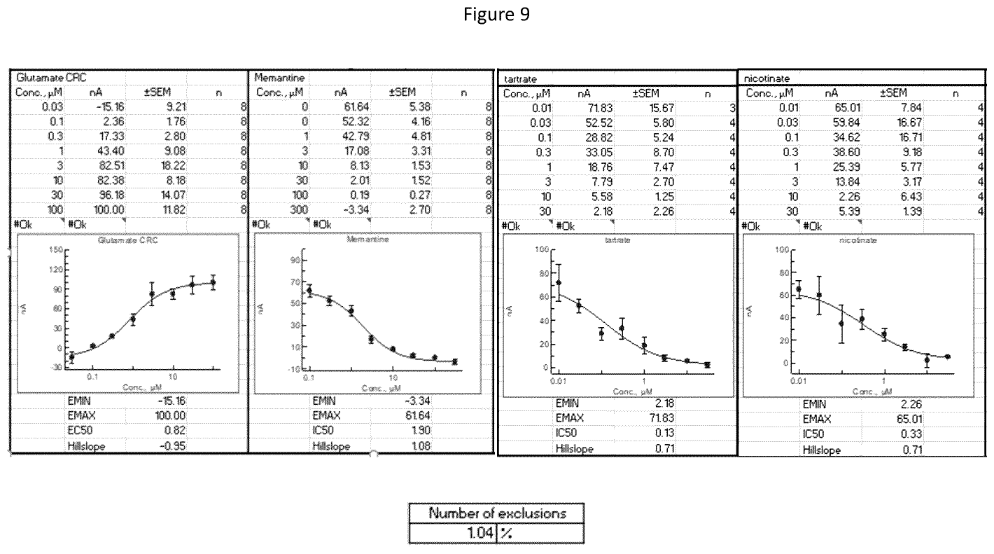

FIG. 10 is an NMR data plot of the ifenprodil nicotinate;

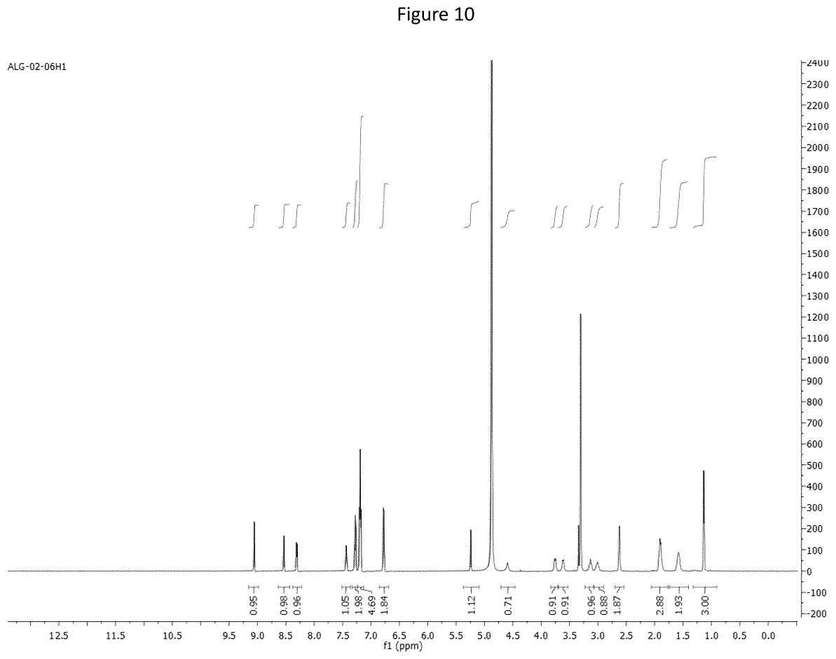

FIG. 11 shows numeric data and plot representation of agonist effects of test articles, vehicle control, and positive controls antagonists. All values were normalized relatively to current elicited with 100 μM glutamate and 50 μM glycine after second application of glutamate. Peak current amplitude measurements are shown;

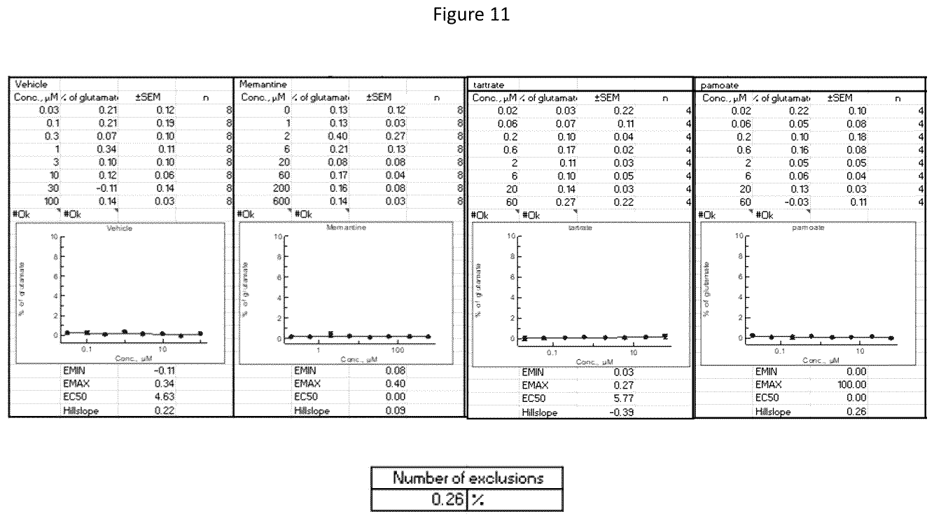

FIG. 12 shows numeric data and plot representation of antagonist effects of test articles and positive controls antagonists co-applied with 2 μM glutamate and 50 μM glycine. All values were normalized relatively to current elicited with 100 μM glutamate and 50 μM glycine. Peak current amplitude measurements are shown.

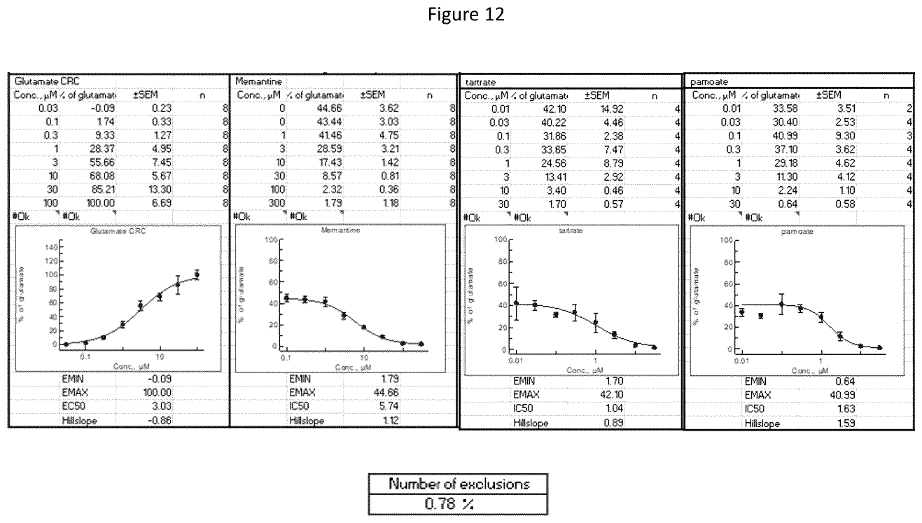

FIG. 13 shows numeric data and plot representation of agonist effects of test articles, vehicle control, and positive control antagonists. All values were normalized relatively to current elicited with 100 μM glutamate and 50 μM glycine during second application of glutamate. Steady state current amplitude measurements are shown;

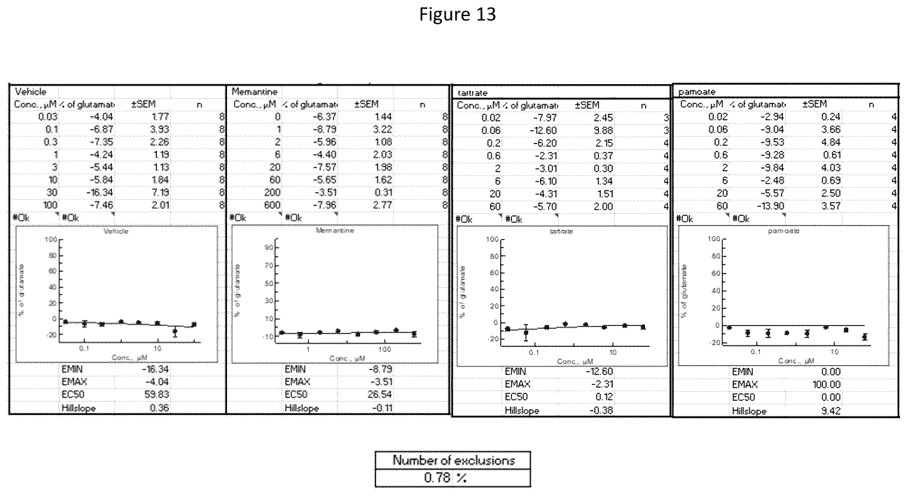

FIG. 14 shows numeric data and plot representation of antagonist effects of test articles and positive controls antagonists co-applied with 2 μM glutamate and 50 μM glycine. All values were normalized relatively to current elicited with 100 μM glutamate and 50 μM glycine. Steady state current amplitude measurements are shown (4-6 seconds after application of glutamate);

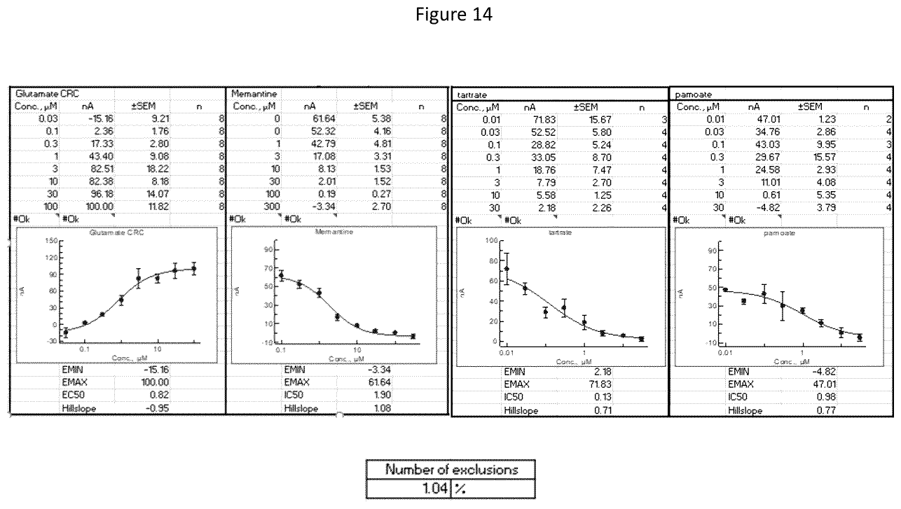

FIG. 15 is an NMR data plot of the ifenprodil pamoate;

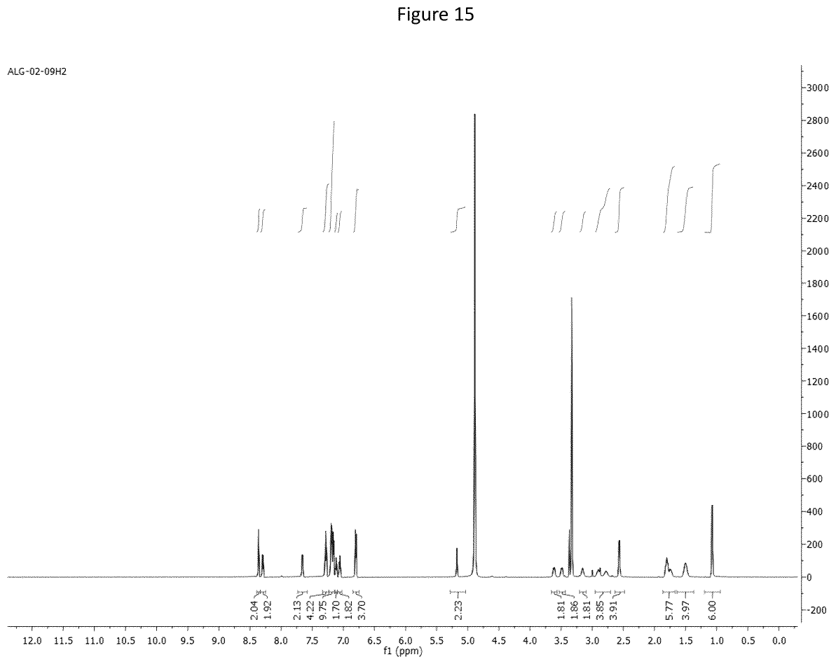

FIG. 16 shows numeric data and plot representation of agonist effects of test articles, vehicle control, and positive controls antagonists. All values were normalized relatively to current elicited with 100 μM glutamate and 50 μM glycine after second application of glutamate. Peak current amplitude measurements are shown;

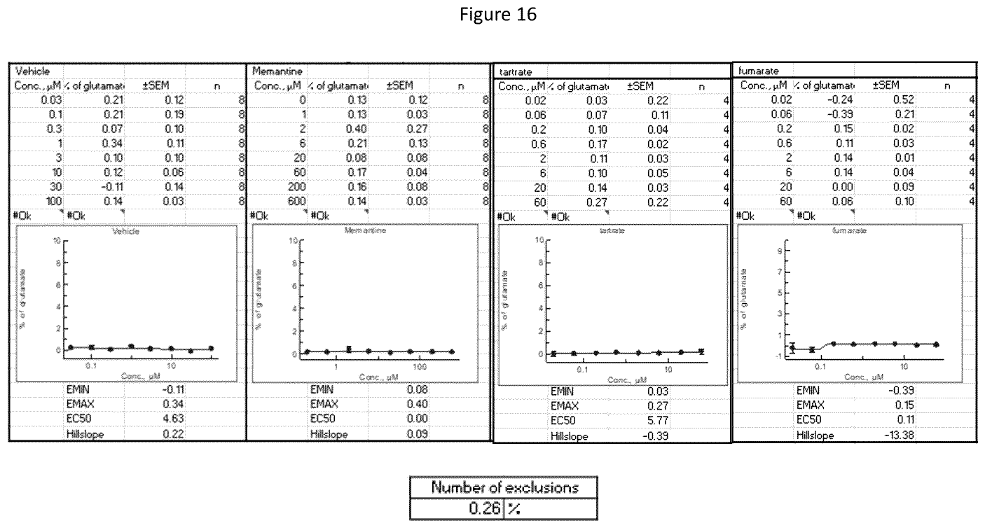

FIG. 17 shows numeric data and plot representation of antagonist effects of test articles and positive controls antagonists co-applied with 2 μM glutamate and 50 μM glycine. All values were normalized relatively to current elicited with 100 μM glutamate and 50 μM glycine. Peak current amplitude measurements are shown.

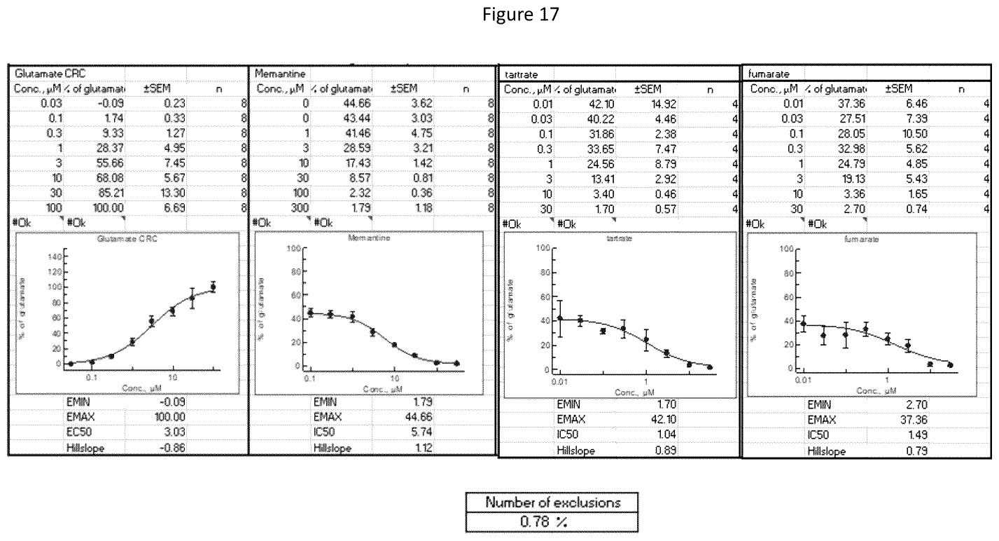

FIG. 18 shows numeric data and plot representation of agonist effects of test articles, vehicle control, and positive control antagonists. All values were normalized relatively to current elicited with 100 μM glutamate and 50 μM glycine during second application of glutamate. Steady state current amplitude measurements are shown;

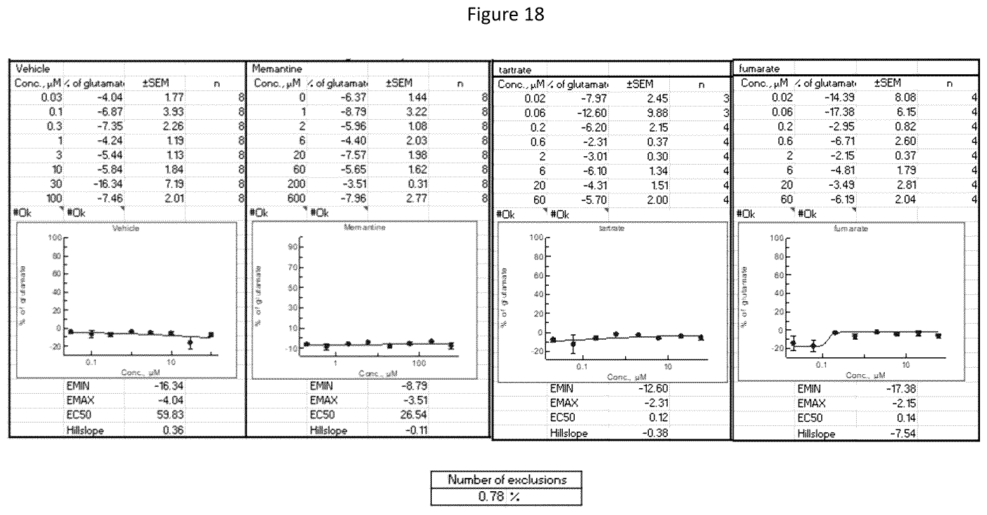

FIG. 19 shows numeric data and plot representation of antagonist effects of test articles and positive controls antagonists co-applied with 2 μM glutamate and 50 μM glycine. All values were normalized relatively to current elicited with 100 μM glutamate and 50 μM glycine. Steady state current amplitude measurements are shown (4-6 seconds after application of glutamate);

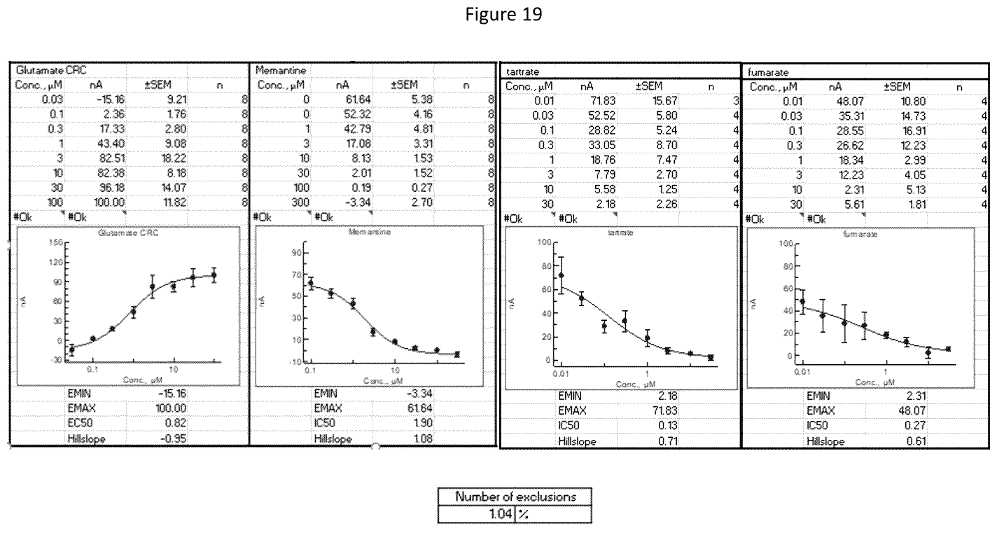

FIG. 20 is an NMR data plot of the ifenprodil fumarate;

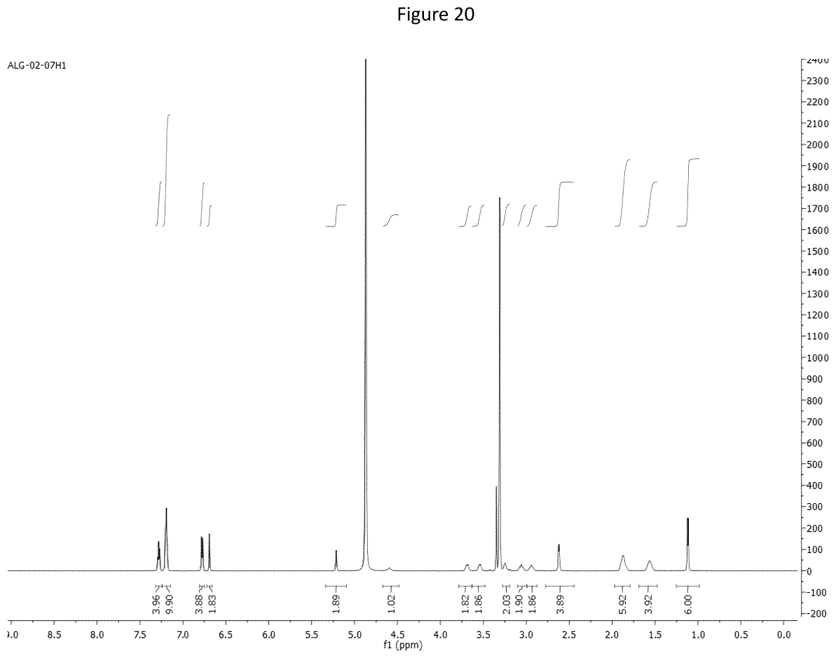

FIG. 21 shows numeric data and plot representation of agonist effects of test articles, vehicle control, and positive controls antagonists. All values were normalized relatively to current elicited with 100 μM glutamate and 50 μM glycine after second application of glutamate. Peak current amplitude measurements are shown;

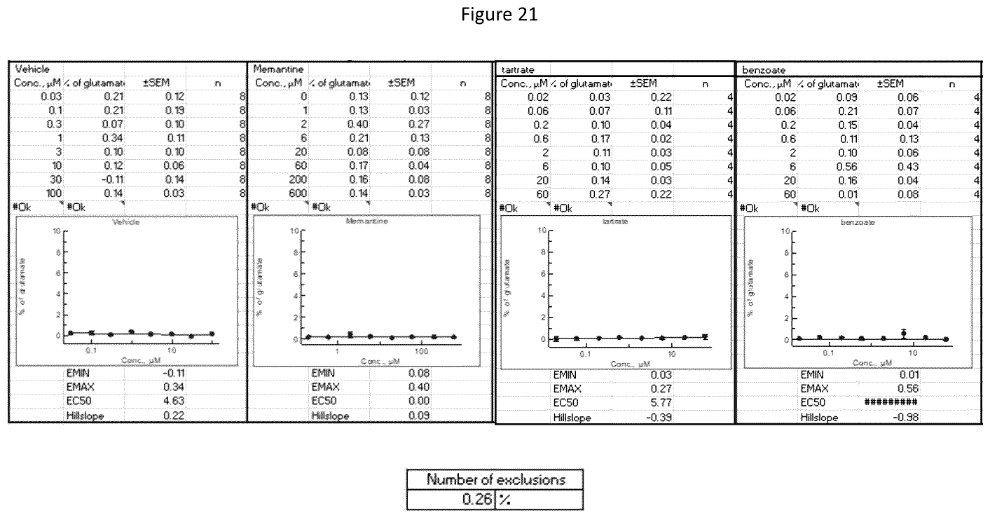

FIG. 22 shows numeric data and plot representation of antagonist effects of test articles and positive controls antagonists co-applied with 2 μM glutamate and 50 μM glycine. All values were normalized relatively to current elicited with 100 μM glutamate and 50 μM glycine. Peak current amplitude measurements are shown.

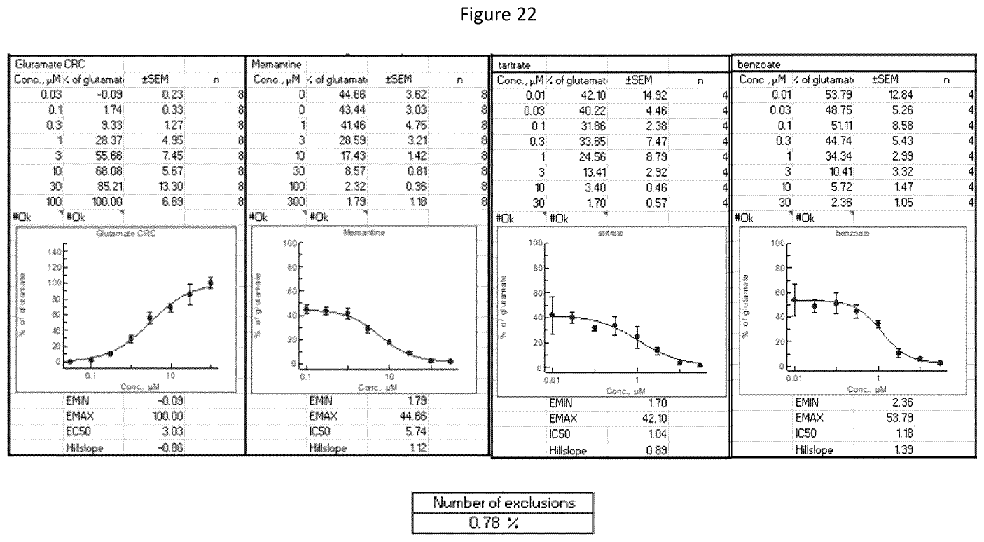

FIG. 23 shows numeric data and plot representation of agonist effects of test articles, vehicle control, and positive control antagonists. All values were normalized relatively to current elicited with 100 μM glutamate and 50 μM glycine during second application of glutamate. Steady state current amplitude measurements are shown;

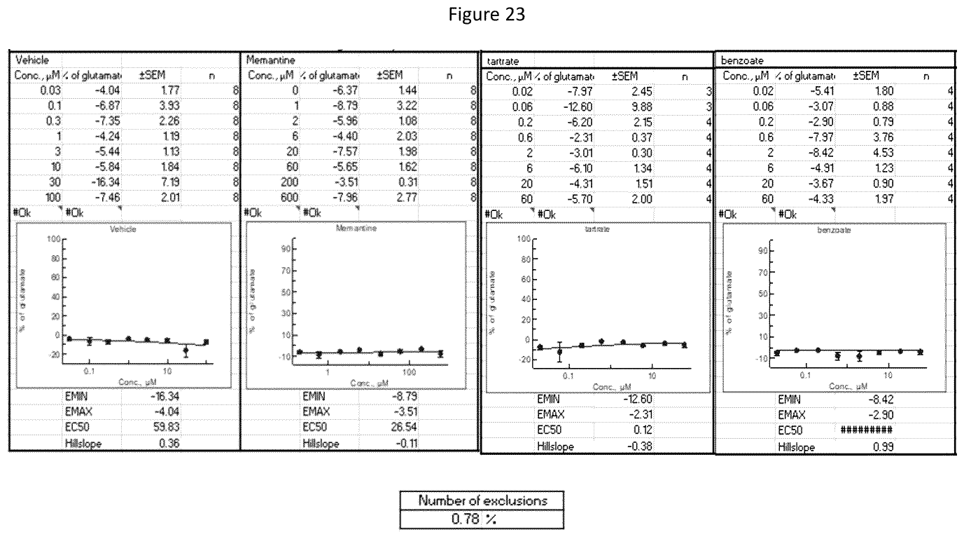

FIG. 24 shows numeric data and plot representation of antagonist effects of test articles and positive controls antagonists co-applied with 2 μM glutamate and 50 μM glycine. All values were normalized relatively to current elicited with 100 μM glutamate and 50 μM glycine. Steady state current amplitude measurements are shown (4-6 seconds after application of glutamate); and

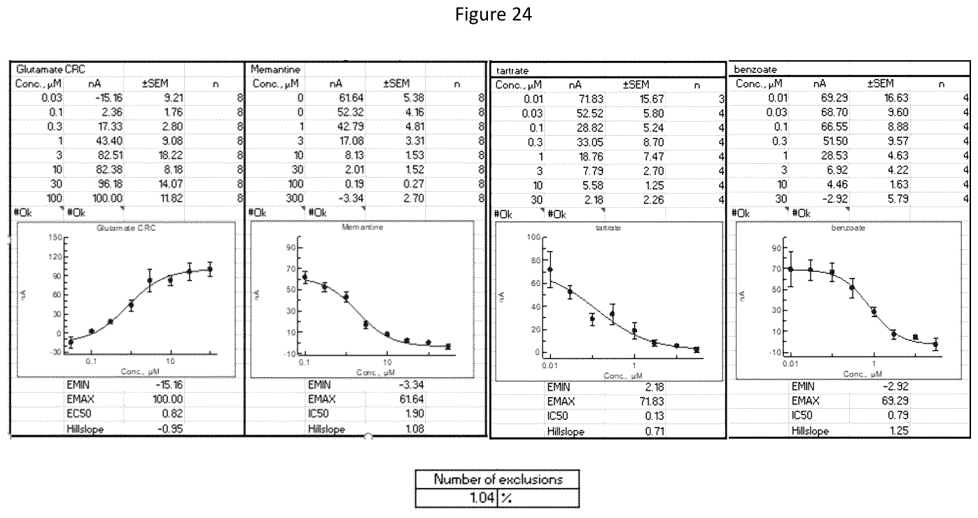

FIG. 25 is an NMR data plot of the ifenprodil benzoate.

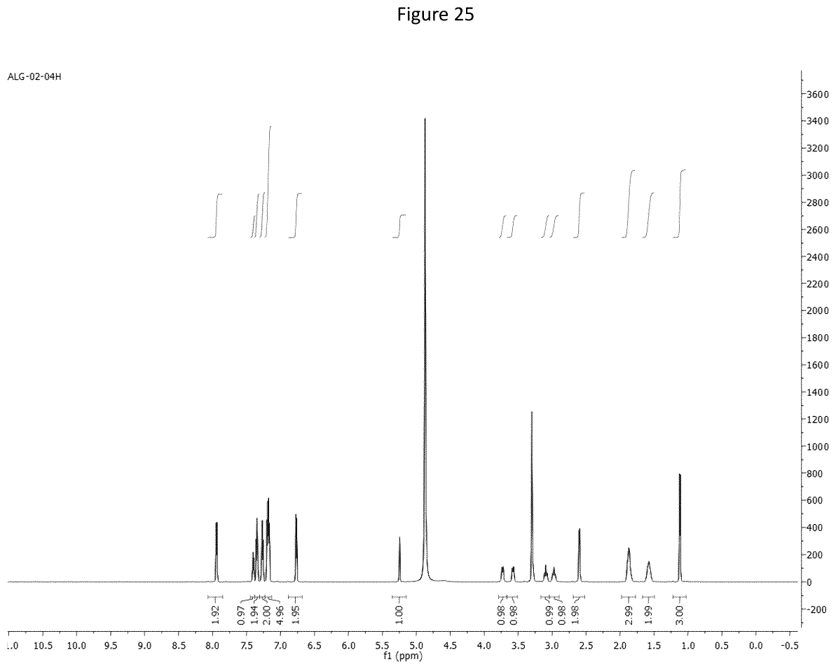

### DETAILED DESCRIPTION

The invention may be more fully appreciated by reference to the following description, including the following glossary of terms and the concluding examples.

Throughout the following description, specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

The present inventors have found that the ifenprodil salts ifenprodil oleate, ifenprodil nicotinate, ifenprodil pamoate, ifenprodil fumarate, and ifenprodil benzoate have a positive effect in the treatment of a number of diseases, disorders, or medical conditions. In some embodiments, administration of a compound of the invention, i.e. ifenprodil oleate, ifenprodil nicotinate, ifenprodil pamoate, ifenprodil fumarate, and ifenprodil benzoate, is effective in preventing the disease, disorder, or medical condition. For example, each of these ifenprodil salts may prevent a disease, disorder, or condition in an individual who may be predisposed to the disease, condition or disorder but does not yet experience or display the pathology or symptomatology of the disease. This is an important finding for treatment and/or prevention of these conditions.

In preferred embodiments, the disease, disorder, or medical condition is selected from: neurologic and psychiatric disorders including, but not limited to: (1) mood disorders and mood affective disorders; (2) neurotic, stress-related and somatoform disorders including anxiety disorders; (3) disorders of psychological development; (4) behavioral syndromes associated with physiological disturbances and physical factors; (5) extrapyramidal and movement disorders; (6) episodic and paroxysmal disorders, epilepsy; (7) pain; (8) forms of neurodegeneration; (9) cerebrovascular diseases, acute and chronic; and any sequelae of cerebrovascular diseases and (10) fibrotic injury, including radiation induced fibrosis.

Examples of mood disorders and mood affective disorders that can be treated according to the certain embodiments include, but are not limited to, bipolar disorder 1 depressed, hypomanic, manic and mixed form; bipolar disorder depressive disorders, such as single depressive episode or recurrent major depressive disorder, minor depressive disorder, treatment-resistant depression, depressive disorder with postpartum onset, depressive disorders with psychotic symptoms; persistent mood disorders, such as cyclothymia, dysthymia, euthymia; and premenstrual dysphoric disorder.

Examples of disorders belonging to the neurotic, stress-related and somatoform disorders that can be treated according to certain embodiments include, but are not limited to, anxiety disorders, general anxiety disorder, panic disorder with or without agoraphobia, specific phobia, social anxiety disorder, chronic anxiety disorders; obsessive compulsive disorder; reaction to severe stress and adjustment disorders, such as post-traumatic stress disorder (PTSD); and other neurotic disorders such as depersonalisation-derealisation syndrome.

Examples of disorders of psychological development that can be treated according certain embodiments include, but are not limited to pervasive developmental disorders, including but not limited to Asperger's syndrome and Rett's syndrome, autistic disorders, childhood autism and overactive disorder associated with mental retardation and stereotyped movements, specific developmental disorder of motor function, and specific developmental disorders of scholastic skills.

Examples of behavioral syndromes associated with physiological disturbances and physical factors according to certain embodiments include, but are not limited to mental and behavioural disorders associated with childbirth, including but not limited to postnatal (postpartum) and prenatal depression; eating disorders, including but not limited to anorexia nervosa, bulimia nervosa, pica, and binge eating disorder.

Examples of extrapyramidal and movement disorders that can be treated according to certain embodiments include, but are not limited to Parkinson's disease; second Parkinsonism, such as postencephalitic Parkinsonism; Parkinsonism comprised in other disorders; Lewy body disease; cerebral palsy; degenerative diseases of the basal ganglia; other extrapyramidal and movement disorders including but not limited to tremor, essential tremor and drug-induced tremor, myoclonus, chorea and drug-induced chorea, drug-induced tics and tics of organic origin, drug-induced acute dystonia, drug-induced tardive dyskinesia, L-dopa-induced dyskinesia; neuroleptic-induced movement disorders including but not limited to neuroleptic malignant syndrome (NMS), neuroleptic induced parkinsonism, neuroleptic-induced early onset or acute dyskinesia, neuroleptic-induced acute dystonia, neuroleptic-induced acute akathisia, neuroleptic-induced tardive dyskinesia, neuroleptic-induced tremor, restless leg syndrome, and Stiff-man syndrome.

Further examples of movement disorders with malfunction and/or degeneration of basal ganglia that can be treated according to certain embodiments include, but are not limited to dystonia including but not limited to focal dystonia, multiple-focal or segmental dystonia, torsion dystonia, hemispheric, generalised and tardive dystonia (induced by psychopharmacological drugs), focal dystonia include cervical dystonia (torticolli), blepharospasm (cramp of the eyelid), appendicular dystonia (cramp in the extremities, like the writer's cramp), and oromandibular dystonia and spasmodic dysphonia (cramp of the vocal cord).

Examples for episodic and paroxysmal disorders that can be treated according to certain embodiments include, but are not limited to epilepsy, including localization-related (focal) (partial) idiopathic epilepsy and epileptic syndromes with seizures of localized onset, localization-related (focal) (partial) symptomatic epilepsy and epileptic syndromes with simple partial seizures, localization-related (focal) (partial) symptomatic epilepsy and epileptic syndromes with complex partial seizures, generalized idiopathic epilepsy and epileptic syndromes including but not limited to myoclonic epilepsy in infancy, neonatal convulsions (familial), childhood absence epilepsy (pyknolepsy), epilepsy with grand mal seizures on awakening, absence epilepsy, myoclonic epilepsy (impulsive petit mal) and nonspecific atonic, clonic, myoclonic, tonic, tonic-clonic epileptic seizures.

Further examples of epilepsy that can be treated according to certain embodiments include, but are not limited to epilepsy with myoclonic absences, myoclonic-astatic seizures, infantile spasms, Lennox-Gastaut syndrome, Salaam attacks, symptomatic early myoclonic encephalopathy, West's syndrome, petit and grand mal seizures, and status epilepticus.

Examples of pain that can be treated according to certain embodiments include, but are not limited to pain disorders related to psychological factors, such as persistent somatoform disorders; acute, chronic and chronic intractable pain, headache; acute and chronic pain related to physiological processes and physical disorders including but not limited to back pain, tooth pain, abdominal pain, low back pain, pain in joints; acute and chronic pain that is related to diseases of the musculoskeletal system and connective tissue including, but not limited to rheumatism, myalgia, neuralgia and fibromyalgia; acute and chronic pain that is related to nerve, nerve root and plexus disorders, such as trigeminal pain, postzoster neuralgia, phantom limb syndrome with pain, carpal tunnel syndrome, lesion of sciatic nerve, diabetic mononeuropathy; acute and chronic pain that is related to polyneuropathies and other disorders of the peripheral nervous system, such as hereditary and idiopathic neuropathy, inflammatory polyneuropathy, polyneuropathy induced by drugs, alcohol or toxic agents, polyneuropathy in neoplastic disease, and diabetic polyneuropathy.

Examples of diseases that are forms of neurodegeneration that can be treated according to certain embodiments include, but are not limited to, acute neurodegeneration, such as intracranial brain injuries, such as stroke, diffuse and local brain injuries, neuronal reperfusion injury, neuronal ischemia, epidural, subdural and subarachnoid haemorrhage, and chronic neurodegeneration, such as Alzheimer's disease, Huntington's disease, multiple sclerosis and ALS. Also included are neuronal infection, and neuronal exposure to a toxic substance.

Examples of cerebrovascular diseases include, but are not limited to, subarachnoid haemorrhage, intracerebral haemorrhage and other nontraumatic intracranial haemorrhage, cerebral infarction, stroke, occlusion and stenosis or precerebral and cerebral arteries, not resulting in cerebral infarction, dissection of cerebral arteries, cerebral aneurysm, cerebral atherosclerosis, progressive vascular leukoencephalopathy, hypertensive encephalopathy, nonpyogenic thrombosis of intracranial venous system, cerebral arteritis, cerebral amyloid angiopathy, and sequelae of cerebrovascular diseases.

Accordingly, the certain embodiments of the invention relate to methods of using the compounds described herein to treat subjects diagnosed with or suffering from a disease, disorder, or condition mediated by NR2B receptor activity, such as: bipolar disorder I depressed, hypomanic, manic and mixed form; bipolar disorder depressive disorders, such as single depressive episode or recurrent major depressive disorder, minor depressive disorder, treatment-resistant depression, depressive disorder with postpartum onset, disruptive mood dysregulation disorder, depressive disorders with psychotic symptoms; persistent mood disorders, such as cyclothymia, dysthymia, euthymia; premenstrual dysphoric disorder; anxiety disorders, general anxiety disorder, panic disorder with or without agoraphobia, specific phobia, social anxiety disorder, chronic anxiety disorders; obsessive compulsive disorder; reaction to severe stress and adjustment disorders, such as post traumatic stress disorder (PTSD); other neurotic disorders such as depersonalisation-derealisation syndrome; pervasive developmental disorders, including but not limited to Asperger's syndrome and Rett's syndrome, autistic disorders, childhood autism and overactive disorder associated with mental retardation and stereotyped movements, specific developmental disorder of motor function, specific developmental disorders of scholastic skills; postnatal (postpartum) and prenatal depression; eating disorders, including but not limited to anorexia nervosa, bulimia nervosa, pica, and binge eating disorder; Parkinson's disease; second Parkinsonism, such as postencephalitic Parkinsonism; Parkinsonism comprised in other disorders; Lewy body disease; degenerative diseases of the basal ganglia; other extrapyramidal and movement disorders including but not limited to tremor, essential tremor and drug-induced tremor, myoclonus, chorea and drug-induced chorea, drug-induced tics and tics of organic origin, drug-induced acute dystonia, drug-induced tardive dyskinesia, L-dopa-induced dyskinesia, cerebral palsy; neuroleptic-induced movement disorders including but not limited to neuroleptic malignant syndrome (NMS), neuroleptic induced parkinsonism, neuroleptic-induced early onset or acute dyskinesia, neuroleptic-induced acute dystonia, neuroleptic-induced acute akathisia, neuroleptic-induced tardive dyskinesia, neuroleptic-induced tremor; restless leg syndrome, Stiff-man syndrome; dystonia including but not limited to focal dystonia, multiple-focal or segmental dystonia, torsion dystonia, hemispheric, generalised and tardive dystonia (induced by psychopharmacological drugs), focal dystonia include cervical dystonia (torticolli), blepharospasm (cramp of the eyelid), appendicular dystonia (cramp in the extremities, like the writer's cramp), oromandibular dystonia and spasmodic dysphonia (cramp of the vocal cord); epilepsy, including localization-related (focal) (partial) idiopathic epilepsy and epileptic syndromes with seizures of localized onset, localization-related (focal) (partial) symptomatic epilepsy and epileptic syndromes with simple partial seizures, localization-related (focal) (partial) symptomatic epilepsy and epileptic syndromes with complex partial seizures, generalized idiopathic epilepsy and epileptic syndromes including but not limited to myoclonic epilepsy in infancy, neonatal convulsions (familial), childhood absence epilepsy (pyknolepsy), epilepsy with grand mal seizures on awakening, absence epilepsy, myoclonic epilepsy (impulsive petit mal) and nonspecific atonic, clonic, myoclonic, tonic, tonic-clonic epileptic seizures; epilepsy with myoclonic absences, myoclonic-astatic seizures, infantile spasms, Lennox-Gastaut syndrome, Salaam attacks, symptomatic early myoclonic encephalopathy, West's syndrome, petit and grand mal seizures; status epilepticus; persistent somatoform disorders; acute, chronic, and chronic intractable pain, headache; acute and chronic pain related to physiological processes and physical disorders including but not limited to back pain, tooth pain, abdominal pain, low back pain, pain in joints; acute and chronic pain that is related to diseases of the musculoskeletal system and connective tissue including, but not limited to rheumatism, myalgia, neuralgia and fibromyalgia; acute and chronic pain that is related to nerve, nerve root and plexus disorders, such as trigeminal pain, postzoster neuralgia, phantom limb syndrome with pain, carpal tunnel syndrome, lesion of sciatic nerve, diabetic mononeuropathy; acute and chronic pain that is related to polyneuropathies and other disorders of the peripheral nervous system, such as hereditary and idiopathic neuropathy, inflammatory polyneuropathy, polyneuropathy induced by drugs, alcohol or toxic agents, polyneuropathy in neoplastic disease, diabetic polyneuropathy; and acute neurodegeneration, such as intracranial brain injuries, such as stroke, diffuse and local brain injuries, neuronal ischemia, neuronal reperfusion injury, epidural, subdural and subarachnoid haemorrhage, and chronic neurodegeneration, such as Alzheimer's disease, Huntington's disease, multiple sclerosis, and ALS; subarachnoid haemorrhage, intracerebral haemorrhage and other nontraumatic intracranial haemorrhage, cerebral infarction, stroke, occlusion and stenosis or precerebral and cerebral arteries, not resulting in cerebral infarction, dissection of cerebral arteries, cerebral aneurysm, cerebral atherosclerosis, progressive vascular leukoencephalopathy, hypertensive encephalopathy, nonpyogenic thrombosis of intracranial venous system, cerebral arteritis, cerebral amyloid angiopathy and sequelae of cerebrovascular diseases; glaucoma and other neuopathies; dementias, vascular demensia, Lewy body dementia, frontotemporal dementia, and HIV-dementia; vertigo and nystagmus; tinnitus; neuropsychiatric systemic lupus erythematosus; disruptive mood dysregulation disorder; schizophrenia spectrum disorder; sleep/wake disorders; Down's Syndrome; Korsakoff's syndrome; neuronal trauma; neuronal infection; exposure to a toxic substance. and neuronal Activation of NR2A-containing NMDA receptors along with modulation of NR2B-containing NMDA receptors in accordance with alternative embodiments of the invention may be implemented so as to achieve particular advantages over previously proposed NMDA receptor antagonism-based therapies. For example, therapies or preventative treatments in accordance with certain embodiments of the invention, in which NR2A-and NR2B-containing NMDA receptors are concomitantly or sequentially modulated, may have a broader therapeutic window than NR2B-containing receptor blockade or modulation therapies alone. In addition, NR2A-containing receptor activation therapies of the invention may be effective not only against NMDA receptor-mediated cell death (primary neuronal injuries), but also in treatment of non-NMDA receptor-mediated cell death (secondary neuronal injuries). In addition to the neuronal injuries caused by acute brain insults such as stroke and brain trauma, utilization of NR2A-containing receptor-dependent pro-survival signaling may also be an effective neuroprotective therapy for a number of chronic neurodegenerative disorders, such Parkinson's disease, Huntington's disease, amyotrophic lateral sclerosis and Alzheimer's disease, where a “slow” NMDA receptor-mediated excitotoxicity has been implicated (Lipton et al., 1994; Ikonomidou et al., 2002; Zoghbi et al., 2000).

The term “treat”, “treatment” or “treating”, as used herein, is intended to refer to administration of an active agent or composition according to an embodiment of the invention to a subject for the purpose of affecting a therapeutic or prophylactic benefit through modulation of NR2B receptor activity. Treating includes reversing, ameliorating, alleviating, inhibiting the progress of, lessening the severity of, or preventing a disease, disorder, or condition, or one or more symptoms of such disease, disorder or condition mediated through modulation of NR2B receptor activity. The term “subject” refers to a mammalian patient in need of such treatment, such as a human.

The term “modulating” encompasses increasing, enhancing, inhibiting, decreasing, suppressing, and the like, generally in a physiologically significant manner.

The expression “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

The term “effective amount” refers to that amount of an active agent or compound that is being administered or that is to be administered, which is sufficient to prevent the disease, disorder, or medical condition, or prevent one or more symptoms of the disease, disorder, or medical condition being treated. In certain embodiments, the term “effective amount” refers to that amount of an active agent or compound that is being administered or that is to be administered, which is sufficient to reduce the risk of the disease, disorder, or medical condition, or one or more symptoms of the disease, disorder, or medical condition. Effective amounts or doses of the compounds of the present invention may be ascertained by routine methods such as modeling, dose escalation studies or clinical trials, and by taking into consideration routine factors, e.g., the mode or route of administration or drug delivery, the pharmacokinetics of the compound, the severity and course of the disease, disorder, or condition, the subject's previous or ongoing therapy, the subject's health status and response to drugs, and the judgment of the treating physician. An example of a dose is in the range of from about 0.001 to about 200 mg of compound per kg of subject's body weight per day, preferably about 0.05 to 100 mg/kg/day, or about 1 to 35 mg/kg/day, in single or divided dosage units (e.g., BID, TID, QID). For a 70-kg human, an illustrative range for a suitable dosage amount is from about 0.05 to about 7 g/day, or about 0.2 to about 2.5 g/day.

In addition, the compounds of the invention may be used in combination with additional active ingredients in the treatment of the above conditions. The additional active ingredients may be co-administered separately for concomitant or sequential administration, or included with such an agent in a pharmaceutical composition according to the invention. In an exemplary embodiment, additional active ingredients are those that are known or discovered to be effective in the treatment of conditions, disorders, or diseases mediated by NR2B activity, including those noted above, such as another NR2B modulator or a compound active against another target associated with the particular condition, disorder, or disease. The combination may serve to increase efficacy (e.g., by including in the combination a compound potentiating the potency or effectiveness of an active agent according to the invention), decrease one or more side effects, or decrease the required dose of the active agent according to the invention.

The compounds of the invention may be used, alone or in combination with one or more additional active ingredients, to formulate pharmaceutical compositions of the invention. A pharmaceutical composition of the invention comprises: (a) an effective amount of at least one compound in accordance with the invention; and (b) a pharmaceutically acceptable excipient.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Also included are disintegrating agents, binding agents, lubricating agents, sweetening agents, flavoring agents, coloring agents and preservative agents. Examples of excipients sodium and calcium carbonate, sodium and calcium phosphate, lactose, starch, sugar, glucose, cellulose derivatives including methyl cellulose, magnesium stearate, polyethylene glycol, mannitol, sorbitol, and the like. Exemplary liquid oral excipients include ethanol, glycerol, water, vegetable oils, and the like. Starch, polyvinyl-pyrrolidone (PVP), sodium starch glycolate, microcrystalline cellulose, and alginic acid are suitable disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating.

Delivery forms of the pharmaceutical compositions containing one or more dosage units of a compound according to the present invention as an active agent may be prepared using suitable pharmaceutical excipients and compounding techniques known or that become available to those skilled in the art. The compositions may be administered in the by a suitable route of delivery, e.g., oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the compounds of the invention can be provided in the form of tablets or capsules, or as a solution, emulsion, or suspension. To prepare the oral compositions, the compounds may be formulated to yield a dosage of, e.g., from about 0.05 to about 100 mg/kg daily, or from about 0.05 to about 35 mg/kg daily, or from about 0.1 to about 10 mg/kg daily. For example, a total daily dosage of about 5 mg to 5 g daily may be accomplished by dosing once, twice, three, or four times per day.

Capsules for oral administration include hard and soft gelatin capsules. To prepare hard gelatin capsules, compounds of the invention may be mixed with a solid, semi-solid, or liquid diluent. Soft gelatin capsules may be prepared by mixing the compound of the invention with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The compounds of this invention may also be administered by non-oral routes. For example, the compositions may be formulated for rectal administration as a suppository. For parenteral use, including intravenous, intramuscular, intraperitoneal, or subcutaneous routes, the compounds of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms are presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses may range from about 1 to 1000 mcg/kg/minute of compound, admixed with a pharmaceutical carrier over a period ranging from several minutes to several days.

For topical administration, the compounds may be mixed with a pharmaceutical carrier at a concentration of about 0.1% to about 10% of drug to vehicle. Another mode of administering the compounds of the invention may utilize a patch formulation to affect transdermal delivery. Compounds of the invention may alternatively be administered in methods of this invention by inhalation, via the nasal or oral routes, e.g., in a spray formulation also containing a suitable carrier.

### EXAMPLES

### Preparation of Ifenprodil Oleate

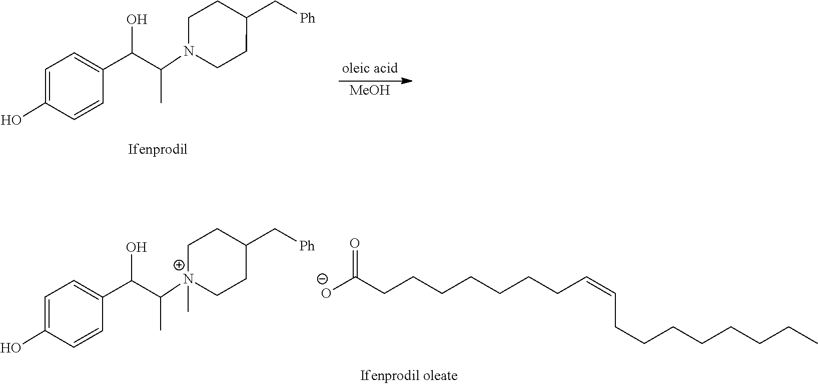 [SMILES: "MeOH" ; CC(C(O)c1ccc(O)cc1)N1CCC(Cc2ccccc2)CC1 ; CC(C(O)c1ccc(O)cc1)[N+]1(C)CCC(Cc2ccccc2)CC1 ; CCCCCCCC/C=C\CCCCCCCC(=O)[O-]]

A 3-neck-25-mL RBF was charged with Ifenprodil (1.5 g, 4.6 mmol, ALG-01-87) and MeOH (7.5 mL, 5 vol). The mixture was stirred at r.t. for 10 min and the morphology turned chunkier. The mixture was then diluted with MeOH (1.5 mL, 1 vol) and heated to 42° C. (internal temperature). The mixture was stirred at 42° C. for 10 min. The oil bath was removed and the mixture was allowed to cool down to the ambient temperature. Meanwhile, oleic acid (1302 mg, 4.6 mmol, TCI O0180 Lot 2AYCI-GA) was diluted in MeOH (1 mL) in a separate flask. Once the internal temperature of Ifenprodil mixture reached below 30° C. (actual temp. 28.3° C.), oleic acid MeOH solution was added in one portion and the mixture turned clear immediately. The solution was cooled to r.t. and then −4° C. in a brine/ice bath. The solution turned cloudy and white precipitate crashed out. The mixture was stirred at −4° C. for 3 h. The solid was collected on a #54 filter paper by filtration and washed by MeOH (3 mL, 2 vol). The material was dried over hi-vac for 24 h to give Ifenprodil oleate (1.66 g, 59% yield, >99% purity by HPLC) as a white solid. The molecular weight was determined to be 607.91.

### In Vitro Effect of Ifenprodil Oleate

Ifenprodil oleate was compared with ifendrodil tartrate (hereafter the “test articles”) in this assay, to assess the in vitro effects, specifically agonist and antagonist ability, of the oleate salt of ifenprodil in comparison to the tartrate salt of ifenprodil. The following patch clamp study investigates these effects on NR1/NR2B ionotropic receptors encoded by the human GRIN1/GRIN2B genes, expressed in HEK293 cells. Compounds were evaluated for functional effects on these ion channel receptors.

### Compounds

The agonist positive control is L-Glutamic acid monosodium salt monohydrate (Sigma-Aldrich), while the antagonist positive controls is memantine chloride (Tocris) and ifenprodil tartrate. The test article is ifenprodil oleate.

### Formulations

Compound solutions are prepared daily. Test article concentrations were prepared by diluting stock solutions into an appropriate HEPES-buffered physiological saline (HB-PS) solution. All test and control solutions will contain 0.6% DMSO, and were sonicated (Model 2510/5510, Branson Ultrasonics, Danbury, CT) at room temperature for at least 20 minutes to facilitate dissolution.

Compound effects were evaluated in 8-point concentration-response format (4replicate wells/concentration). The compound formulations were loaded in a 384-well compound plate using an automated liquid handling system (Assist Plus, Integra).

Compounds were prepared in stock solutions in vials of appropriate volume to test the compounds at the concentrations listed in Table 1 below.

TABLE 1

| Compound Name | Test concentrations, μM |
|---|---|
| Glutamate | 0.001, 0.03, 0.1, 0.3, 1, 3, 10, 30 |
| Memantine | 0.001, 0.03, 0.1, 0.3, 1, 3, 10, 30 |
| Ifenprodil tartrate | 0.001, 0.03, 0.1, 0.3, 1, 3, 10, 30 |
| Ifenprodil oleate | 0.001, 0.03, 0.1, 0.3, 1, 3, 10, 30 |

### Testing System and Method

The assay was carried out using an HTS electrophysiology-based approach with the Syncropatch 384pe automated patch clamp system (Nanion), while using HEK293 cells that have been transfected with human GRIN1/GRIN2B genes to express NR1/NR2B ionotropic receptors

HEK293 cells were transfected with the appropriate ion channel or receptor cDNA(s) encoding NR1 and NR2B. Stable transfectants were selected using the G418 and Zeocin-resistance genes incorporated into the expression plasmid. Selection pressure was maintained with G418 and Zeocin in the culture medium. Cells were cultured in Dulbecco's Modified Eagle Medium/Nutrient Mixture F-12 (D-MEM/F-12) supplemented with 10% fetal bovine serum, 100 U/ml penicillin G sodium, 100 ug/mL streptomycin sulfate, 100 ug/mL Zeocin, 5 ug/mL blasticidin and 500 ug/mL G418.

Before testing, cells in culture dishes were washed twice with DPBS solution. Immediately before testing, the cells were washed in HB-PS containing 10 mM CaCl₂ to improve sealing.

Compounds were evaluated in 8-point concentration-response format (4 replicate wells/concentration). The compound formulations were loaded in e.g. a 384-well compound plate and placed in the Syncropatch plate well.

To monitor the sensitivity the assay, for each plate, the standard antagonist positive control article (Memantine, Tocris) was applied at 8 half log concentrations (range 0.1-300 μM); n=4, where n=the number of replicates per concentration. The agonist positive control (L-glutamate) was applied at eight (8) concentrations (0.03-100 μM; n=4, where n=the number of replicates) together with 50 μM glycine.

2× concentration of test compounds and antagonist control was pre-applied 2 minutes before application of L-glutamate/glycine mixed with 1× concentration of test compound. Glutamate and glycine was applied to naive cells (n=4, where n=the number of replicate wells/concentration) via a multi-channel pipettor.

### Electrophysiological Procedures

a) Intracellular solution (mM): 50 mM CsCl, 90 mM CsF, 2 mM MgCl₂, 5 mM EGTA, 10 mM HEPES. Adjust to pH 7.2 with CsOH. This solution was prepared in batches and stored refrigerated. In preparation for a recording session, the intracellular solution was loaded into the intracellular compartment of the multi holes (8×) planar electrode.
b) Extracellular solution, HB-PS (composition in mM): NaCl, 137; KCl, 1.0; CaCl₂, 2; HEPES, 10; Glucose, 10; pH adjusted to 7.4 with NaOH (refrigerated until use).
c) Holding potential: −70 mV, potential during agonist/antagonist application: −70 mV.
Test Compound Administration: The application consisted of the addition of 20 μL of 1× concentrated test article solution and agonist at 40 μL/s (1 second total application time).
Positive Control Agonist: 0.03-100 μM L-glutamate (8 concentration concentration-response, half log scale) and 50 μM glycine, stimulation with 2 μM glutamate, 50 μM glycine.
Positive control antagonist: 0.1-300 μM memantine (8 concentration dose-response, half log scale dilution) co-applied with 2 μM glutamate and 50 μM glycine.

### Recording Procedure

a) Extracellular buffer was loaded into the multi holes plate wells (40 μL per well). Cell suspension was pipetted into the wells (20 μL per well) of the multi holes planar electrode.
b) Whole-cell recording configuration was established via patch perforation with membrane currents recorded by on-board patch clamp amplifiers.
c) Two recording (scans) were performed: First, during test compound and control application alone (2× concentration) and second, during co-application of test compound or antagonist control (1× concentration) with agonist stimulation (˜EC₅₀ L-glutamate) to detect antagonist effects of the compounds.

### Screenpatch Data Analysis

Activation was calculated in two ways based on the following measurements:
1. peak current amplitudes, and
2. current amplitude 4-5 seconds after agonist addition.

Data acquisition was performed via the FLIPR Control software that is supplied with the FLIPR System (MDS-AT) and data was analyzed using Microsoft Excel™ (Microsoft™ Corp., Redmond, WA). Concentration-response data was filled to a Hill equation of the following form:

$$RESPONSE = Ba se + \frac{Max - Base}{1 + {( \frac{xhalf}{x} )}^{rate}}$$

where Base is the response in the absence of test article (vehicle treatment), Max is the maximum response at high concentrations, xhalf is the EC₅₀, or IC₅₀, the concentration of test compound producing either half-maximal activation or inhibition, and rate is the Hill coefficient. Nonlinear least squares fits were made assuming a simple binding model. If appropriate, fits were weighted by the standard deviation. No assumptions about the fit parameters were made; the fit parameters were determined by algorithm. Raw reduced data was analyzed using Microsoft Excel Office 365 ProPlus (Microsoft™ Corp., Redmond, WA) and XLfit (Excel addon, Copyright© IDBS 2016).

For each plate, a Z′ Factor and Signal Window was calculated in accordance with published online Assay Guidance Manual: https://www.ncbi.nlm.nih.gov/books/NBK83783/#htsvalidation.Bridging_Studies_for_Assay

Preferably, the raw data will meet the following acceptance criteria:
The Z′ factor must be ≥0.4
SW factor must be ≥2.

### Results

Glutamate (plus 50 μM glycine) activated receptors with EC₅₀ of 3.03 μM for PCA and EC₅₀ of 0.82 μM for SSC measurements. All values were calculated relatively to current produced by 100 μM glutamate (E_{MAX} set as 100%, % of control).

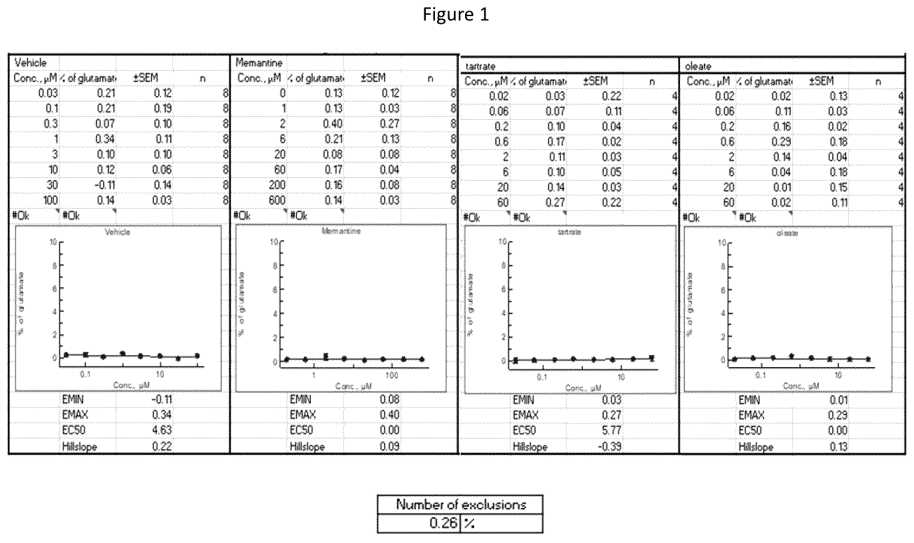

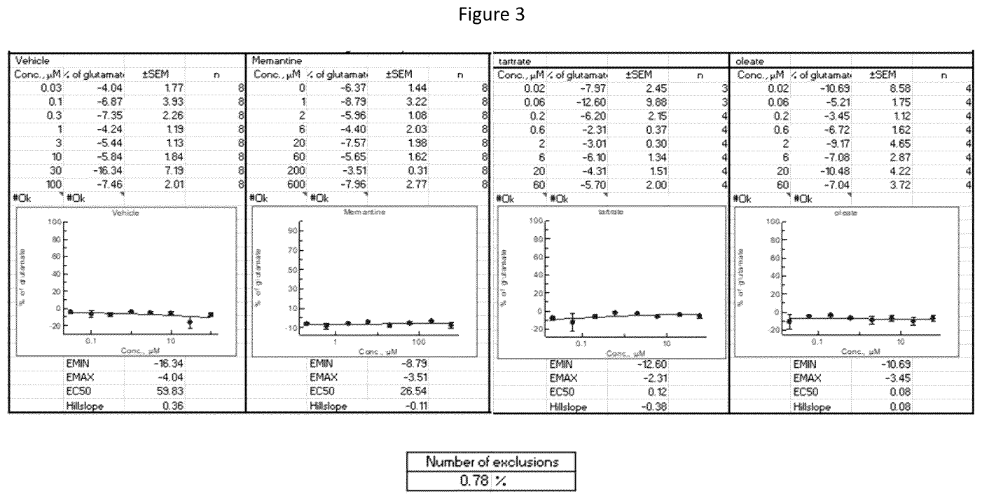

The agonist effect of test articles and the positive controls were examined. Table 2 below shows peak current amplitude measurements (PCA) and Table 3—steady state current measurements (SSC). Further data showing any agonist effect can be seen in FIGS. 1 and 3, which show peak current amplitude and steady state current amplitude measurements, respectively.

TABLE 2
Agonist and antagonist effects of test articles on NR1/NR2B
receptors: peak current amplitude measurements.

| | | NR1/NR2B | | | NR1/NR2B | |
|---|---|---|---|---|---|---|
| | | PCA agonist | | | PCA antagonist | |
| | Compound | EMAX, | EC50, | | IC50, | |
| # | ID | % | μM | HillSlope | μM | HillSlope |
| 1 | tartrate | — | — | — | 1.04 | 0.89 |
| 2 | oleate | — | — | — | 1.17 | 0.79 |
| 3 | Memantine | — | — | — | 5.74 | 1.12 |
| 4 | Glutamate | 100.00 | 3.03 | −0.86 | — | — |
| | CRC | | | | | |

TABLE 3
Agonist and antagonist effects of test articles on NR1/NR2B
receptors: steady state current amplitude measurements

| | | NR1/NR2B | | | NR1/NR2B | |
|---|---|---|---|---|---|---|
| | | PCA agonist | | | PCA antagonist | |
| | Compound | EMAX, | EC50, | | IC50, | |
| # | ID | % | μM | HillSlope | μM | HillSlope |
| 1 | tartrate | — | — | — | 0.31 | 0.79 |
| 2 | oleate | — | — | — | 0.49 | 0.86 |
| 3 | Memantine | — | — | — | 1.90 | 1.08 |
| 4 | Glutamate | 100.00 | 0.82 | −0.95 | — | — |
| | CRC | | | | | |

Antagonist activity of test articles was examined after stimulation of receptors with 2 μM glutamate and 50 μM glycine in the presence of increasing concentrations of test articles. Two measurements were performed, first was a peak current amplitude and second was a steady state current between 4^{th} and 6^{th} seconds following agonist application. It should be noted that previous studies have shown that open channel blockers (uncompetitive type of inhibition) are more potent at blocking steady state current as compared to peak current amplitude.

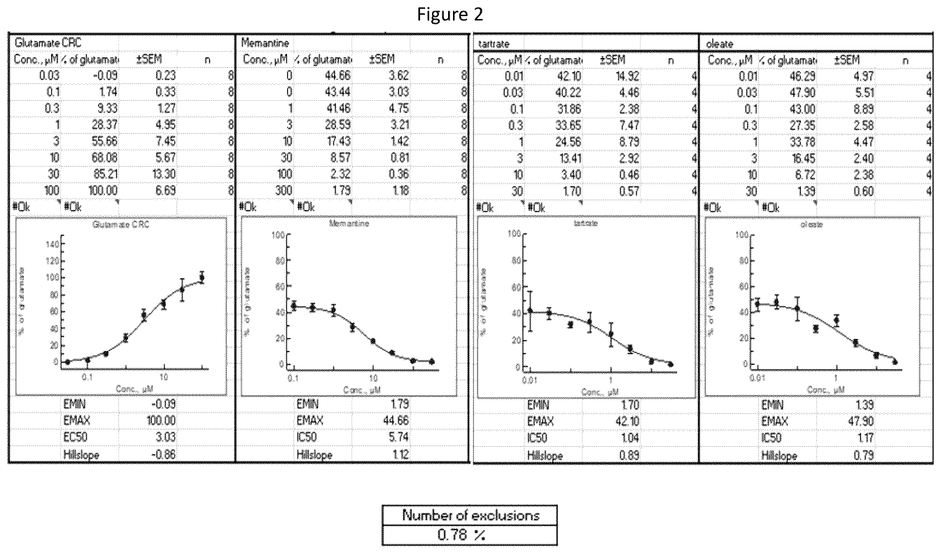

Tables 2 and 3 show the calculated IC₅₀ and Hillslope values, while further data showing antagonist effects of the controls and test articles can be seen in FIGS. 2 (PCA measurements) and 4 (SSC measurements).

All controls articles inhibited NR1/NR2B NMDA receptors having an IC₅₀ in the range of 1.04-1.17 μM for PCA measurements and 0.31-0.49 μM range for SSC measurements. SSC was inhibited to a somewhat greater extent.

Reference negative allosteric modulator, ifenprodil, inhibited NR1/NR2B NMDA receptors with an IC₅₀ of 1.67 μM and 0.54 μM for PCA and SSC measurements respectively.

Reference antagonist, memantine, inhibited NR1/NR2B NMDA receptors with an IC₅₀ of 5.74 μM and 1.90 μM for PCA and SSC measurements respectively.

### Conclusion

In conclusion, ifenprodil oleate inhibits glutamate and glycine activation of NR1/NR2B receptors in vitro at a level comparable to ifenprodil tartrate and to the control antagonists when PCA and SSC measurements were taken.

### Preparation of Ifenprodil Nicotinate

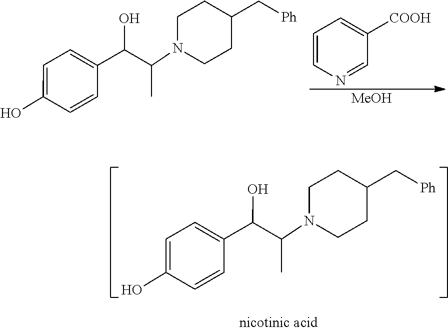

A 3-neck-25-mL RBF was charged with Ifenprodil (1.5 g, 4.6 mmol, ALG-02-02) and MeOH (7.5 mL, 5 vol). The mixture was stirred at r.t. for 10 min and the morphology turned chunkier. The mixture was then diluted with MeOH (1.5 mL, 1 vol) and heated to 60° C. (internal temperature). Meanwhile, nicotinic acid (567 mg, 4.6 mmol, Sigma-Aldrich N785-0 Lot 0720DL) was added in MeOH (17 mL, 30 vol) in a separate flask.

The mixture was heated to 60° C. and stirred until a clear solution was obtained. Nicotinic acid/MeOH solution was then added to Ifenprodil/MeOH mixture at 60° C. and the mixture turned clear immediately. The solution was stirred at 60° C. for 10 min. The oil bath was then removed and the solution was allowed to cool to the ambient temperature and stirred there for 1 h. The solution was then stirred in a brine/ice bath for 1 h. No precipitate formed during this process. MeOH was then removed under vacuum. The oil residue was dried over hi-vac to give Ifenprodil nicotinate (1.92 g, 93% yield) as a white solid.

### In Vitro Effect of Ifenprodil Nicotinate

Ifenprodil nicotinate was compared with ifendrodil tartrate (hereafter the “test articles”) in this assay, to assess the in vitro effects, specifically agonist and antagonist ability, of the nicotinate salt of ifenprodil in comparison to the tartrate salt of ifenprodil. The following patch clamp study investigates these effects on NR1/NR2B ionotropic receptors encoded by the human GRIN1/GRIN2B genes, expressed in HEK293 cells. Compounds were evaluated for functional effects on these ion channel receptors.

### Compounds

The agonist positive control is L-Glutamic acid monosodium salt monohydrate (Sigma-Aldrich), while the antagonist positive controls is memantine chloride (Tocris) and ifenprodil tartrate. The test article is ifenprodil nicotinate.

### Formulations

Compound solutions are prepared daily. Test article concentrations were prepared by diluting stock solutions into an appropriate HEPES-buffered physiological saline (HB-PS) solution. All test and control solutions will contain 0.6% DMSO, and were sonicated (Model 2510/5510, Branson Ultrasonics, Danbury, CT) at room temperature for at least 20 minutes to facilitate dissolution.

Compound effects were evaluated in 8-point concentration-response format (4 replicate wells/concentration). The compound formulations were loaded in a 384-well compound plate using an automated liquid handling system (Assist Plus, Integra).

Compounds were prepared in stock solutions in vials of appropriate volume to test the compounds at the concentrations listed in Table 4 below.

TABLE 4

| Compound Name | Test concentrations, μM |
|---|---|
| Glutamate | 0.001, 0.03, 0.1, 0.3, 1, 3, 10, 30 |
| Memantine | 0.001, 0.03, 0.1, 0.3, 1, 3, 10, 30 |
| Ifenprodil tartrate | 0.001, 0.03, 0.1, 0.3, 1, 3, 10, 30 |
| Ifenprodil nicotinate | 0.001, 0.03, 0.1, 0.3, 1, 3, 10, 30 |

### Testing System And Method

The assay was carried out using an HTS electrophysiology-based approach with the Syncropatch 384pe automated patch clamp system (Nanion), while using HEK293 cells that have been transfected with human GRIN1/GRIN2B genes to express NR1/NR2B ionotropic receptors

HEK293 cells were transfected with the appropriate ion channel or receptor cDNA(s) encoding NR1 and NR2B. Stable transfectants were selected using the G418 and Zeocin-resistance genes incorporated into the expression plasmid. Selection pressure was maintained with G418 and Zeocin in the culture medium. Cells were cultured in Dulbecco's Modified Eagle Medium/Nutrient Mixture F-12 (D-MEM/F-12) supplemented with 10% fetal bovine serum, 100 U/mL penicillin G sodium, 100 ug/mL streptomycin sulfate, 100 ug/mL Zeocin, 5 ug/mL blasticidin and 500 ug/mL G418.

Before testing, cells in culture dishes were washed twice with DPBS solution. Immediately before testing, the cells were washed in HB-PS containing 10 mM CaCl₂ to improve sealing.

Compounds were evaluated in 8-point concentration-response format (4 replicate wells/concentration). The compound formulations were loaded in e.g. a 384-well compound plate and placed in the Syncropatch plate well.

To monitor the sensitivity the assay, for each plate, the standard antagonist positive control article (Memantine, Tocris) was applied at 8 half log concentrations (range 0.1-300 μM); n=4, where n=the number of replicates per concentration. The agonist positive control (L-glutamate) was applied at eight (8) concentrations (0.03-100 μM; n=4, where n=the number of replicates) together with 50 μM glycine.

2× concentration of test compounds and antagonist control was pre-applied 2 minutes before application of L-glutamate/glycine mixed with 1× concentration of test compound. Glutamate and glycine was applied to naive cells (n=4, where n=the number of replicate wells/concentration) via a multi-channel pipettor.

### Electrophysiological Procedures

a) Intracellular solution (mM): 50 mM CsCl, 90 mM CsF, 2 mM MgCl₂, 5 mM EGTA, 10 mM HEPES. Adjust to pH 7.2 with CsOH. This solution was prepared in batches and stored refrigerated. In preparation for a recording session, the intracellular solution was loaded into the intracellular compartment of the multi holes (8×) planar electrode.
b) Extracellular solution, HB-PS (composition in mM): NaCl, 137; KCl, 1.0; CaCl₂, 2; HEPES, 10; Glucose, 10; pH adjusted to 7.4 with NaOH (refrigerated until use).
c) Holding potential: −70 mV, potential during agonist/antagonist application: −70 mV.
Test Compound Administration: The application consisted of the addition of 20 μL of 1× concentrated test article solution and agonist at 40 μL/s (1 second total application time).
Positive Control Agonist: 0.03-100 μM L-glutamate (8 concentration concentration-response, half log scale) and 50 μM glycine, stimulation with 2 μM glutamate, 50 μM glycine.
Positive control antagonist: 0.1-300 μM memantine (8 concentration dose-response, half log scale dilution) co-applied with 2 μM glutamate and 50 μM glycine.

### Recording Procedure

a) Extracellular buffer was loaded into the multi holes plate wells (40 μL per well). Cell suspension was pipetted into the wells (20 μL per well) of the multi holes planar electrode.
b) Whole-cell recording configuration was established via patch perforation with membrane currents recorded by on-board patch clamp amplifiers.
c) Two recording (scans) were performed: First, during test compound and control application alone (2× concentration) and second, during co-application of test compound or antagonist control (1× concentration) with agonist stimulation (˜EC₅₀ L-glutamate) to detect antagonist effects of the compounds.

### Screenpatch Data Analysis

Activation was calculated in two ways based on the following measurements:
1. peak current amplitudes, and
2. current amplitude 4-5 seconds after agonist addition.

Data acquisition was performed via the FLIPR Control software that is supplied with the FLIPR System (MDS-AT) and data was analyzed using Microsoft Excel™ (Microsoft™ Corp., Redmond, WA). Concentration-response data was fitted to a Hill equation of the following form:

$$RESPONSE = Ba se + \frac{Max - Base}{1 + {( \frac{xhalf}{x} )}^{rate}}$$

where Base is the response in the absence of test article (vehicle treatment), Max is the maximum response at high concentrations, xhalf is the EC₅₀, or IC₅₀, the concentration of test compound producing either half-maximal activation or inhibition, and rate is the Hill coefficient. Nonlinear least squares fits were made assuming a simple binding model. If appropriate, fits were weighted by the standard deviation. No assumptions about the fit parameters were made; the fit parameters were determined by algorithm. Raw reduced data was analyzed using Microsoft Excel Office 365 ProPlus (Microsoft™ Corp., Redmond, WA) and XLfit (Excel addon, Copyright© IDBS 2016).

For each plate, a Z′ Factor and Signal Window was calculated in accordance with published online Assay Guidance Manual: https://www.ncbi.nlm.nih.gov/books/NBK83783/#htsvalidation.Bridging_Studies_for_Assay

Preferably, the raw data will meet the following acceptance criteria:
The Z′ factor must be ≥0.4
SW factor must be ≥2.

### Results

Glutamate (plus 50 μM glycine) activated receptors with EC₅₀ of 3.03 μM for PCA and EC₅₀ of 0.82 μM for SSC measurements. All values were calculated relatively to current produced by 100 μM glutamate (E_{MAX} set as 100%, % of control).

The agonist effect of test articles and the positive controls were examined. Table 5 below shows peak current amplitude measurements (PCA) and Table 6—steady state current measurements (SSC). Further data showing any agonist effect can be seen in FIGS. 1 and 3, which show peak current amplitude and steady state current amplitude measurements, respectively.

TABLE 5
Agonist and antagonist effects of test articles on NR1/NR2B
receptors: peak current amplitude measurements.

| | | NR1/NR2B | | | NR1/NR2B | |
|---|---|---|---|---|---|---|
| | | PCA agonist | | | PCA antagonist | |
| | Compound | EMAX, | EC50, | | IC50, | |
| # | ID | % | μM | HillSlope | μM | HillSlope |
| 1 | tartrate | — | — | — | 1.04 | 0.89 |
| 2 | nicotinate | — | — | — | 1.16 | 0.71 |
| 3 | Memantine | — | — | — | 5.74 | 1.12 |
| 4 | Glutamate | 100.00 | 3.03 | −0.86 | — | — |
| | CRC | | | | | |

TABLE 6
Agonist and antagonist effects of test articles on NR1/NR2B
receptors: steady state current amplitude measurements

| | | NR1/NR2B | | | NR1/NR2B | |
|---|---|---|---|---|---|---|
| | | PCA agonist | | | PCA antagonist | |
| | Compound | EMAX, | EC50, | | IC50, | |
| # | ID | % | μM | HillSlope | μM | HillSlope |
| 1 | tartrate | — | — | — | 0.31 | 0.79 |
| 2 | nicotinate | — | — | — | 0.33 | 0.71 |
| 3 | Memantine | — | — | — | 1.90 | 1.08 |
| 4 | Glutamate | 100.00 | 0.82 | −0.95 | — | — |
| | CRC | | | | | |

Antagonist activity of test articles was examined after stimulation of receptors with 2 μM glutamate and 50 μM glycine in the presence of increasing concentrations of test articles. Two measurements were performed, first was a peak current amplitude and second was a steady state current between 4^{th} and 6^{th} seconds following agonist application. It should be noted that previous studies have shown that open channel blockers (uncompetitive type of inhibition) are more potent at blocking steady state current as compared to peak current amplitude.

Tables 5 and 6 show the calculated IC₅₀ and Hillslope values, while further data showing antagonist effects of the controls and test articles can be seen in FIGS. 2 (PCA measurements) and 4 (SSC measurements).

All controls articles inhibited NR1/NR2B NMDA receptors having an IC₅₀ in the range of 1.04-1.17 μM for PCA measurements and 0.31-0.49 μM range for SSC measurements. SSC was inhibited to a somewhat greater extent.

Reference negative allosteric modulator, ifenprodil, inhibited NR1/NR2B NMDA receptors with an IC₅₀ of 1.67 μM and 0.54 μM for PCA and SSC measurements respectively.

Reference antagonist, memantine, inhibited NR1/NR2B NMDA receptors with an IC₅₀ of 5.74 μM and 1.90 μM for PCA and SSC measurements respectively.

### Conclusion

In conclusion, ifenprodil nicotinate inhibits glutamate and glycine activation of NR1/NR2B receptors in vitro at a level comparable to ifenprodil tartrate and to the control antagonists when PCA and SSC measurements were taken.

### Preparation of Ifenprodil Pamoate

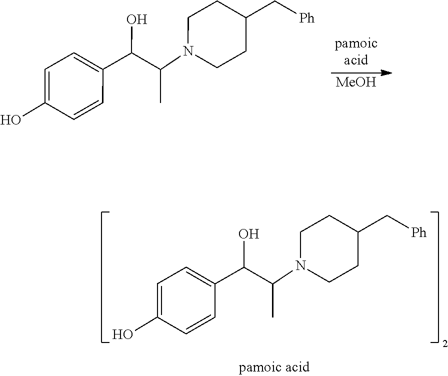

A 3-neck-25-mL RBF was charged with Ifenprodil (1.5 g, 4.6 mmol, ALG-02-02) and MeOH (7.5 mL, 5 vol). The mixture was stirred at r.t. for 10 min and the morphology turned chunkier. The mixture was then diluted with MeOH (1.5 mL, 1 vol) and heated to 42° C. (internal temperature). The mixture was stirred at 42° C. for 10 min. The oil bath was removed and the mixture was allowed to cool down to the ambient temperature. Once the internal temperature of Ifenprodil mixture reached below 30° C. (actual temp. 28.5° C.), pamoic acid (895 mg, 2.3 mmol, Sigma-Aldrich 45150 Lot BCBF1746V) was added in one portion. The mixture was then heated to 35° C. and stirred for 10 min. The oil bath was then removed and the solution was allowed to cool to the ambient temperature. The solution was then cooled in a brine/ice bath and stirred for 30 min. No precipitate formed during this process. MeOH was then removed under vacuum. The yellow residue was dried over hi-vac to give Ifenprodil hemipamoate (2.34 g, 98% yield) as a yellow solid.

### In Vitro Effect of Ifenprodil Pamoate

Ifenprodil pamoate was compared with ifendrodil tartrate (hereafter the “test articles”) in this assay, to assess the in vitro effects, specifically agonist and antagonist ability, of the pamoate salt of ifenprodil in comparison to the tartrate salt of ifenprodil. The following patch clamp study investigates these effects on NR1/NR2B ionotropic receptors encoded by the human GRIN1/GRIN2B genes, expressed in HEK293 cells. Compounds were evaluated for functional effects on these ion channel receptors.

### Compounds

The agonist positive control is L-Glutamic acid monosodium salt monohydrate (Sigma-Aldrich), while the antagonist positive controls is memantine chloride (Tocris) and ifenprodil tartrate. The test article is ifenprodil pamoate.

Formulations

Compound solutions are prepared daily. Test article concentrations were prepared by diluting stock solutions into an appropriate HEPES-buffered physiological saline (HB-PS) solution. All test and control solutions will contain 0.6% DMSO, and were sonicated (Model 2510/5510, Branson Ultrasonics, Danbury, CT) at room temperature for at least 20 minutes to facilitate dissolution.

Compound effects were evaluated in 8-point concentration-response format (4 replicate wells/concentration). The compound formulations were loaded in a 384-well compound plate using an automated liquid handling system (Assist Plus, Integra).

Compounds were prepared in stock solutions in vials of appropriate volume to test the compounds at the concentrations listed in Table 7 below.

TABLE 7

| Compound Name | Test concentrations, μM |
|---|---|
| Glutamate | 0.001, 0.03, 0.1, 0.3, 1, 3, 10, 30 |
| Memantine | 0.001, 0.03, 0.1, 0.3, 1, 3, 10, 30 |
| Ifenprodil tartrate | 0.001, 0.03, 0.1, 0.3, 1, 3, 10, 30 |
| Ifenprodil pamoate | 0.001, 0.03, 0.1, 0.3, 1, 3, 10, 30 |

### Testing System And Method

The assay was carried out using an HTS electrophysiology-based approach with the Syncropatch 384pe automated patch clamp system (Nanion), while using HEK293cells that have been transfected with human GRIN1/GRIN2B genes to express NR1/NR2B ionotropic receptors

HEK293 cells were transfected with the appropriate ion channel or receptor cDNA(s) encoding NR1 and NR2B. Stable transfectants were selected using the G418 and Zeocin-resistance genes incorporated into the expression plasmid. Selection pressure was maintained with G418 and Zeocin in the culture medium. Cells were cultured in Dulbecco's Modified Eagle Medium/Nutrient Mixture F-12 (D-MEM/F-12) supplemented with 10% fetal bovine serum, 100 U/ml penicillin G sodium, 100 ug/mL streptomycin sulfate, 100 ug/mL Zeocin, 5 ug/mL blasticidin and 500 ug/mL G418.

Before testing, cells in culture dishes were washed twice with DPBS solution. Immediately before testing, the cells were washed in HB-PS containing 10 mM CaCl₂ to improve sealing.

Compounds were evaluated in 8-point concentration-response format (4 replicate wells/concentration). The compound formulations were loaded in e.g. a 384-well compound plate and placed in the Syncropatch plate well.

To monitor the sensitivity the assay, for each plate, the standard antagonist positive control article (Memantine, Tocris) was applied at 8 half log concentrations (range 0.1-300 μM); n=4, where n=the number of replicates per concentration. The agonist positive control (L-glutamate) was applied at eight (8) concentrations (0.03-100 μM; n=4, where n=the number of replicates) together with 50 μM glycine.

2× concentration of test compounds and antagonist control was pre-applied 2 minutes before application of L-glutamate/glycine mixed with 1× concentration of test compound. Glutamate and glycine was applied to naive cells (n=4, where n=the number of replicate wells/concentration) via a multi-channel pipettor.

### Electrophysiological Procedures

a) Intracellular solution (mM): 50 mM CsCl, 90 mM CsF, 2 mM MgCl₂, 5 mM EGTA, 10 mM HEPES. Adjust to pH 7.2 with CsOH. This solution was prepared in batches and stored refrigerated. In preparation for a recording session, the intracellular solution was loaded into the intracellular compartment of the multi holes (8×) planar electrode.
b) Extracellular solution, HB-PS (composition in mM): NaCl, 137; KCl, 1.0; CaCl₂, 2; HEPES, 10; Glucose, 10; pH adjusted to 7.4 with NaOH (refrigerated until use).
c) Holding potential: −70 mV, potential during agonist/antagonist application: −70 mV.
Test Compound Administration: The application consisted of the addition of 20 μL of 1× concentrated test article solution and agonist at 40 μL/s (1 second total application time).
Positive Control Agonist: 0.03-100 μM L-glutamate (8 concentration concentration-response, half log scale) and 50 μM glycine, stimulation with 2 μM glutamate, 50 μM glycine.
Positive control antagonist: 0.1-300 μM memantine (8 concentration dose-response, half log scale dilution) co-applied with 2 μM glutamate and 50 μM glycine.

### Recording Procedure

a) Extracellular buffer was loaded into the multi holes plate wells (40 μL per well). Cell suspension was pipetted into the wells (20 μL per well) of the multi holes planar electrode.
b) Whole-cell recording configuration was established via patch perforation with membrane currents recorded by on-board patch clamp amplifiers.
c) Two recording (scans) were performed: First, during test compound and control application alone (2× concentration) and second, during co-application of test compound or antagonist control (1× concentration) with agonist stimulation (˜EC₅₀ L-glutamate) to detect antagonist effects of the compounds.

### Screenpatch Data Analysis

Activation was calculated in two ways based on the following measurements:
1. peak current amplitudes, and
2. current amplitude 4-5 seconds after agonist addition.

Data acquisition was performed via the FLIPR Control software that is supplied with the FLIPR System (MDS-AT) and data was analyzed using Microsoft Excel™ (Microsoft™ Corp., Redmond, WA). Concentration-response data was fitted to a Hill equation of the following form:

$$RESPONSE = Ba se + \frac{Max - Base}{1 + {( \frac{xhalf}{x} )}^{rate}}$$

where Base is the response in the absence of test article (vehicle treatment), Max is the maximum response at high concentrations, xhalf is the EC₅₀, or IC₅₀, the concentration of test compound producing either half-maximal activation or inhibition, and rate is the Hill coefficient. Nonlinear least squares fits were made assuming a simple binding model. If appropriate, fits were weighted by the standard deviation. No assumptions about the fit parameters were made; the fit parameters were determined by algorithm. Raw reduced data was analyzed using Microsoft Excel Office 365 ProPlus (Microsoft™ Corp., Redmond, WA) and XLfit (Excel addon, Copyright© IDBS 2016).

For each plate, a Z′ Factor and Signal Window was calculated in accordance with published online Assay Guidance Manual: https://www.ncbi.nlm.nih.gov/books/NBK83783/#htsvalidation.Bridging_Studies_for_Assay

Preferably, the raw data will meet the following acceptance criteria:
The Z′ factor must be ≥0.4
SW factor must be ≥2.

### Results

Glutamate (plus 50 μM glycine) activated receptors with EC₅₀ of 3.03 μM for PCA and EC₅₀ of 0.82 μM for SSC measurements. All values were calculated relatively to current produced by 100 μM glutamate (E_{MAX} set as 100%, % of control).

The agonist effect of test articles and the positive controls were examined. Table 8 below shows peak current amplitude measurements (PCA) and Table 9—steady state current measurements (SSC). Further data showing any agonist effect can be seen in FIGS. 1 and 3, which show peak current amplitude and steady state current amplitude measurements, respectively.

TABLE 8
Agonist and antagonist effects of test articles on NR1/NR2B
receptors: peak current amplitude measurements.

| | | NR1/NR2B | | | NR1/NR2B | |
|---|---|---|---|---|---|---|
| | | PCA agonist | | | PCA antagonist | |
| | Compound | EMAX, | EC50, | | IC50, | |
| # | ID | % | μM | HillSlope | μM | HillSlope |
| 1 | tartrate | — | — | — | 1.04 | 0.89 |
| 2 | pamoate | — | — | — | 1.63 | 1.59 |
| 3 | Memantine | — | — | — | 5.74 | 1.12 |
| 4 | Glutamate | 100.00 | 3.03 | −0.86 | — | — |
| | CRC | | | | | |

TABLE 9
Agonist and antagonist effects of test articles on NR1/NR2B
receptors: steady state current amplitude measurements

| | | NR1/NR2B | | | NR1/NR2B | |
|---|---|---|---|---|---|---|
| | | PCA agonist | | | PCA antagonist | |
| | Compound | EMAX, | EC50, | | IC50, | |
| # | ID | % | μM | HillSlope | μM | HillSlope |
| 1 | tartrate | — | — | — | 0.31 | 0.79 |
| 2 | pamoate | — | — | — | 0.98 | 0.77 |
| 3 | Memantine | — | — | — | 1.90 | 1.08 |
| 4 | Glutamate | 100.00 | 0.82 | −0.95 | — | — |
| | CRC | | | | | |

Antagonist activity of test articles was examined after stimulation of receptors with 2 μM glutamate and 50 μM glycine in the presence of increasing concentrations of test articles. Two measurements were performed, first was a peak current amplitude and second was a steady state current between 4^{th} and 6^{th} seconds following agonist application. It should be noted that previous studies have shown that open channel blockers (uncompetitive type of inhibition) are more potent at blocking steady state current as compared to peak current amplitude.

Tables 8 and 9 show the calculated IC₅₀ and Hillslope values, while further data showing antagonist effects of the controls and test articles can be seen in FIGS. 2 (PCA measurements) and 4 (SSC measurements).

All controls articles inhibited NR1/NR2B NMDA receptors having an IC₅₀ in the range of 1.04-1.17 μM for PCA measurements and 0.31-0.49 μM range for SSC measurements. SSC was inhibited to a somewhat greater extent.

Reference negative allosteric modulator, ifenprodil, inhibited NR1/NR2B NMDA receptors with an IC₅₀ of 1.67 μM and 0.54 μM for PCA and SSC measurements respectively.

Reference antagonist, memantine, inhibited NR1/NR2B NMDA receptors with an IC₅₀ of 5.74 μM and 1.90 μM for PCA and SSC measurements respectively.

### Conclusion

In conclusion, ifenprodil pamoate inhibits glutamate and glycine activation of NR1/NR2B receptors in vitro at a level comparable to ifenprodil tartrate and to the control antagonists when PCA and SSC measurements were taken.

### Preparation of Ifenprodil Fumarate

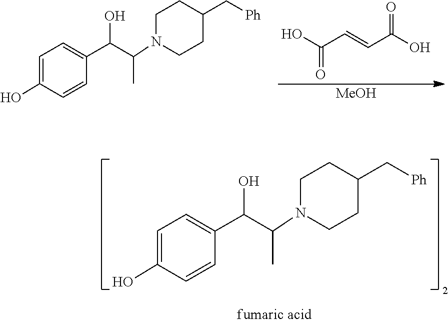

A 3-neck-25-mL RBF was charged with Ifenprodil (1.5 g, 4.6 mmol, ALG-02-02) and MeOH (7.5 mL, 5 vol). The mixture was stirred at r.t. for 10 min and the morphology turned chunkier. The mixture was then diluted with MeOH (1.5 mL, 1 vol) and heated to 42° C. (internal temperature). The mixture was stirred at 42° C. for 10 min. The oil bath was removed and the mixture was allowed to cool down to the ambient temperature. Once the internal temperature of Ifenprodil mixture reached below 30° C. (actual temp. 27.5° C.), fumaric acid (267 mg, 2.3 mmol, Sigma-Aldrich 24074-5 Lot 06709EV) was added in one portion and the mixture turned clear immediately. The solution was stirred at r.t. for 20 and then cooled in a brine/ice bath. White precipitate formed after 15 min. The mixture was stirred in the brine/ice bath for 2 h. The solid was collected on a #54 filter paper by filtration and washed by MeOH (3 mL, 2 vol). The material was dried over hi-vac for 24 h to give Ifenprodil hemifumarate (1.64 g) as a white solid. The solid was triturated in methyl ethyl ketone to reduce/remove MeOH residue.

### In Vitro Effect of Ifenprodil Fumarate

Ifenprodil fumarate was compared with ifendrodil tartrate (hereafter the “test articles”) in this assay, to assess the in vitro effects, specifically agonist and antagonist ability, of the fumarate salt of ifenprodil in comparison to the tartrate salt of ifenprodil. The following patch clamp study investigates these effects on NR1/NR2B ionotropic receptors encoded by the human GRIN1/GRIN2B genes, expressed in HEK293 cells. Compounds were evaluated for functional effects on these ion channel receptors.

### Compounds

The agonist positive control is L-Glutamic acid monosodium salt monohydrate (Sigma-Aldrich), while the antagonist positive controls is memantine chloride (Tocris) and ifenprodil tartrate. The test article is ifenprodil fumarate.

### Formulations

Compound solutions are prepared daily. Test article concentrations were prepared by diluting stock solutions into an appropriate HEPES-buffered physiological saline (HB-PS) solution. All test and control solutions will contain 0.6% DMSO, and were sonicated (Model 2510/5510, Branson Ultrasonics, Danbury, CT) at room temperature for at least 20 minutes to facilitate dissolution.

Compound effects were evaluated in 8-point concentration-response format (4 replicate wells/concentration). The compound formulations were loaded in a 384-well compound plate using an automated liquid handling system (Assist Plus, Integra).

Compounds were prepared in stock solutions in vials of appropriate volume to test the compounds at the concentrations listed in Table 10 below.

TABLE 10

| Compound Name | Test concentrations, μM |
|---|---|
| Glutamate | 0.001, 0.03, 0.1, 0.3, 1, 3, 10, 30 |
| Memantine | 0.001, 0.03, 0.1, 0.3, 1, 3, 10, 30 |
| Ifenprodil tartrate | 0.001, 0.03, 0.1, 0.3, 1, 3, 10, 30 |
| Ifenprodil fumarate | 0.001, 0.03, 0.1, 0.3, 1, 3, 10, 30 |

### Testing System And Method

The assay was carried out using an HTS electrophysiology-based approach with the Syncropatch 384pe automated patch clamp system (Nanion), while using HEK293 cells that have been transfected with human GRIN1/GRIN2B genes to express NR1/NR2B ionotropic receptors

HEK293 cells were transfected with the appropriate ion channel or receptor cDNA(s) encoding NR1 and NR2B. Stable transfectants were selected using the G418 and Zeocin-resistance genes incorporated into the expression plasmid. Selection pressure was maintained with G418 and Zeocin in the culture medium. Cells were cultured in Dulbecco's Modified Eagle Medium/Nutrient Mixture F-12 (D-MEM/F-12) supplemented with 10% fetal bovine serum, 100 U/mL penicillin G sodium, 100 ug/mL streptomycin sulfate, 100 ug/mL Zeocin, 5 ug/mL blasticidin and 500 ug/mL G418.

Before testing, cells in culture dishes were washed twice with DPBS solution. Immediately before testing, the cells were washed in HB-PS containing 10 mM CaCl₂ to improve sealing.

Compounds were evaluated in 8-point concentration-response format (4 replicate wells/concentration). The compound formulations were loaded in e.g. a 384-well compound plate and placed in the Syncropatch plate well.

To monitor the sensitivity the assay, for each plate, the standard antagonist positive control article (Memantine, Tocris) was applied at 8 half log concentrations (range 0.1-300 μM); n=4, where n=the number of replicates per concentration. The agonist positive control (L-glutamate) was applied at eight (8) concentrations (0.03-100 μM; n=4, where n=the number of replicates) together with 50 μM glycine.

2× concentration of test compounds and antagonist control was pre-applied 2 minutes before application of L-glutamate/glycine mixed with 1× concentration of test compound. Glutamate and glycine was applied to naive cells (n=4, where n=the number of replicate wells/concentration) via a multi-channel pipettor.

### Electrophysiological Procedures

a) Intracellular solution (mM): 50 mM CsCl, 90 mM CsF, 2 mM MgCl₂, 5 mM EGTA, 10 mM HEPES. Adjust to pH 7.2 with CsOH. This solution was prepared in batches and stored refrigerated. In preparation for a recording session, the intracellular solution was loaded into the intracellular compartment of the multi holes (8×) planar electrode.
b) Extracellular solution, HB-PS (composition in mM): NaCl, 137; KCl, 1.0; CaCl₂, 2; HEPES, 10; Glucose, 10; pH adjusted to 7.4 with NaOH (refrigerated until use).
c) Holding potential: −70 mV, potential during agonist/antagonist application: −70 mV.
Test Compound Administration: The application consisted of the addition of 20 μL of 1× concentrated test article solution and agonist at 40 μL/s (1 second total application time).
Positive Control Agonist: 0.03-100 μM L-glutamate (8 concentration concentration-response, half log scale) and 50 μM glycine, stimulation with 2 μM glutamate, 50 μM glycine.
Positive control antagonist: 0.1-300 μM memantine (8 concentration dose-response, half log scale dilution) co-applied with 2 μM glutamate and 50 μM glycine.

### Recording Procedure

a) Extracellular buffer was loaded into the multi holes plate wells (40 μL per well). Cell suspension was pipetted into the wells (20 μL per well) of the multi holes planar electrode.
b) Whole-cell recording configuration was established via patch perforation with membrane currents recorded by on-board patch clamp amplifiers.
c) Two recording (scans) were performed: First, during test compound and control application alone (2× concentration) and second, during co-application of test compound or antagonist control (1× concentration) with agonist stimulation (˜EC₅₀ L-glutamate) to detect antagonist effects of the compounds.

### Screenpatch Data Analysis

Activation was calculated in two ways based on the following measurements:
1. peak current amplitudes, and
2. current amplitude 4-5 seconds after agonist addition.

Data acquisition was performed via the FLIPR Control software that is supplied with the FLIPR System (MDS-AT) and data was analyzed using Microsoft Excel™ (Microsoft™ Corp., Redmond, WA). Concentration-response data was fitted to a Hill equation of the following form:

$$RESPONSE = Ba se + \frac{Max - Base}{1 + {( \frac{xhalf}{x} )}^{rate}}$$

where Base is the response in the absence of test article (vehicle treatment), Max is the maximum response at high concentrations, xhalf is the EC₅₀, or IC₅₀, the concentration of test compound producing either half-maximal activation or inhibition, and rate is the Hill coefficient. Nonlinear least squares fits were made assuming a simple binding model. If appropriate, fits were weighted by the standard deviation. No assumptions about the fit parameters were made; the fit parameters were determined by algorithm. Raw reduced data was analyzed using Microsoft Excel Office 365 ProPlus (Microsoft™ Corp., Redmond, WA) and XLfit (Excel addon, Copyright© IDBS 2016).

For each plate, a Z′ Factor and Signal Window was calculated in accordance with published online Assay Guidance Manual: https://www.ncbi.nlm.nih.gov/books/NBK83783/#htsvalidation.Bridging_Studies_for_Assay

Preferably, the raw data will meet the following acceptance criteria:
The Z′ factor must be ≥0.4
SW factor must be ≥2.

### Results

Glutamate (plus 50 μM glycine) activated receptors with EC₅₀ of 3.03 μM for PCA and EC₅₀ of 0.82 μM for SSC measurements. All values were calculated relatively to current produced by 100 μM glutamate (E_{MAX} set as 100%, % of control).

The agonist effect of test articles and the positive controls were examined. Table 11 below shows peak current amplitude measurements (PCA) and Table 12—steady state current measurements (SSC). Further data showing any agonist effect can be seen in FIGS. 1 and 3, which show peak current amplitude and steady state current amplitude measurements, respectively.

TABLE 11
Agonist and antagonist effects of test articles on NR1/NR2B
receptors: peak current amplitude measurements.

| | | NR1/NR2B | | | NR1/NR2B | |
|---|---|---|---|---|---|---|
| | | PCA agonist | | | PCA antagonist | |
| | Compound | EMAX, | EC50, | | IC50, | |
| # | ID | % | μM | HillSlope | μM | HillSlope |
| 1 | tartrate | — | — | — | 1.04 | 0.89 |
| 2 | fumarate | — | — | — | 1.49 | 0.79 |
| 3 | Memantine | — | — | — | 5.74 | 1.12 |
| 4 | Glutamate | 100.00 | 3.03 | −0.86 | — | — |
| | CRC | | | | | |

TABLE 12
Agonist and antagonist effects of test articles on NR1/NR2B
receptors: steady state current amplitude measurements

| | | NR1/NR2B | | | NR1/NR2B | |
|---|---|---|---|---|---|---|
| | | PCA agonist | | | PCA antagonist | |
| | Compound | EMAX, | EC50, | | IC50, | |
| # | ID | % | μM | HillSlope | μM | HillSlope |
| 1 | tartrate | — | — | — | 0.31 | 0.79 |
| 2 | fumarate | — | — | — | 0.27 | 0.61 |
| 3 | Memantine | — | — | — | 1.90 | 1.08 |
| 4 | Glutamate | 100.00 | 0.82 | −0.95 | — | — |
| | CRC | | | | | |

Antagonist activity of test articles was examined after stimulation of receptors with 2 μM glutamate and 50 μM glycine in the presence of increasing concentrations of test articles. Two measurements were performed, first was a peak current amplitude and second was a steady state current between 4^{th} and 6^{th} seconds following agonist application. It should be noted that previous studies have shown that open channel blockers (uncompetitive type of inhibition) are more potent at blocking steady state current as compared to peak current amplitude.

Tables 11 and 12 show the calculated IC₅₀ and Hillslope values, while further data showing antagonist effects of the controls and test articles can be seen in FIGS. 2 (PCA measurements) and 4 (SSC measurements).

All controls articles inhibited NR1/NR2B NMDA receptors having an IC₅₀ in the range of 1.04-1.17 μM for PCA measurements and 0.31-0.49 μM range for SSC measurements. SSC was inhibited to a somewhat greater extent.

Reference negative allosteric modulator, ifenprodil, inhibited NR1/NR2B NMDA receptors with an IC₅₀ of 1.67 μM and 0.54 μM for PCA and SSC measurements respectively.

Reference antagonist, memantine, inhibited NR1/NR2B NMDA receptors with an IC₅₀ of 5.74 μM and 1.90 μM for PCA and SSC measurements respectively.

### Conclusion

In conclusion, ifenprodil fumarate inhibits glutamate and glycine activation of NR1/NR2B receptors in vitro at a level comparable to ifenprodil tartrate and to the control antagonists when PCA and SSC measurements were taken.

### Preparation of Ifenprodil Benzoate

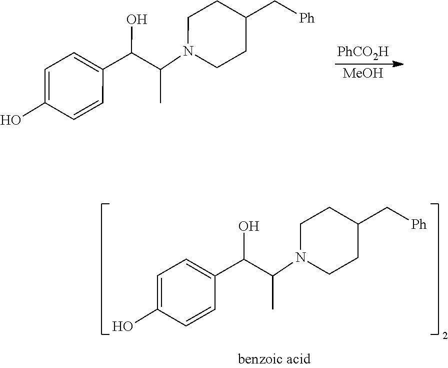

A 3-neck-25-mL RBF was charged with Ifenprodil (1.5 g, 4.6 mmol, ALG-01-87) and MeOH (7.5 mL, 5 vol). The mixture was stirred at r.t. for 10 min and the morphology turned chunkier. The mixture was then diluted with MeOH (1.5 mL, 1 vol) and heated to 42° C. (internal temperature). The mixture was stirred at 42° C. for 10 min. The oil bath was removed and the mixture was allowed to cool down to the ambient temperature. Meanwhile, benzoic acid (563 mg, 4.6 mmol, Mallinckrodt Lot WVN2) was dissolved in MeOH (1.7 mL, 3 vol) in a separate flask. Once the internal temperature of Ifenprodil mixture reached below 30° C. (actual temp. 28.3° C.), benzoic acid/MeOH solution was added in one portion and the mixture turned clear immediately. The solution was stirred at r.t. for 1 h and at −4° C. for 1 h in a brine/ice bath. The cold bath was then removed, the solution turned cloudy and white precipitate crashed out. The flask was placed back in the brine/ice cold bath and the mixture was stirred for 3 h. The solid was collected on a #54 filter paper by filtration and washed by MeOH (3 mL, 2 vol). The material was dried over hi-vac for 24 h to give Ifenprodil benzoate (1.12 g, 54% yield) as a white solid.

### In Vitro Effect of Ifenprodil Benzoate

Ifenprodil benzoate was compared with ifendrodil tartrate (hereafter the “test articles”) in this assay, to assess the in vitro effects, specifically agonist and antagonist ability, of the benzoate salt of ifenprodil in comparison to the tartrate salt of ifenprodil. The following patch clamp study investigates these effects on NR1/NR2B ionotropic receptors encoded by the human GRIN1/GRIN2B genes, expressed in HEK293 cells. Compounds were evaluated for functional effects on these ion channel receptors.

### Compounds

The agonist positive control is L-Glutamic acid monosodium salt monohydrate (Sigma-Aldrich), while the antagonist positive controls is memantine chloride (Tocris) and ifenprodil tartrate. The test article is ifenprodil benzoate.

### Formulations

Compound solutions are prepared daily. Test article concentrations were prepared by diluting stock solutions into an appropriate HEPES-buffered physiological saline (HB-PS) solution. All test and control solutions will contain 0.6% DMSO, and were sonicated (Model 2510/5510, Branson Ultrasonics, Danbury, CT) at room temperature for at least 20 minutes to facilitate dissolution.

Compound effects were evaluated in 8-point concentration-response format (4 replicate wells/concentration). The compound formulations were loaded in a 384-well compound plate using an automated liquid handling system (Assist Plus, Integra).

Compounds were prepared in stock solutions in vials of appropriate volume to test the compounds at the concentrations listed in Table 13 below.

TABLE 13

| Compound Name | Test concentrations, μM |
|---|---|
| Glutamate | 0.001, 0.03, 0.1, 0.3, 1, 3, 10, 30 |
| Memantine | 0.001, 0.03, 0.1, 0.3, 1, 3, 10, 30 |
| Ifenprodil tartrate | 0.001, 0.03, 0.1, 0.3, 1, 3, 10, 30 |
| Ifenprodil benzoate | 0.001, 0.03, 0.1, 0.3, 1, 3, 10, 30 |

### Testing System And Method

The assay was carried out using an HTS electrophysiology-based approach with the Syncropatch 384pe automated patch clamp system (Nanion), while using HEK293 cells that have been transfected with human GRIN1/GRIN2B genes to express NR1/NR2B ionotropic receptors

HEK293 cells were transfected with the appropriate ion channel or receptor cDNA(s) encoding NR1 and NR2B. Stable transfectants were selected using the G418 and Zeocin-resistance genes incorporated into the expression plasmid. Selection pressure was maintained with G418 and Zeocin in the culture medium. Cells were cultured in Dulbecco's Modified Eagle Medium/Nutrient Mixture (D-MEM/F-12) supplemented with 10% fetal bovine serum, 100 U/mL penicillin G sodium, 100 ug/mL streptomycin sulfate, 100 ug/mL Zeocin, 5 ug/mL blasticidin and 500 ug/mL G418.

Before testing, cells in culture dishes were washed twice with DPBS solution. Immediately before testing, the cells were washed in HB-PS containing 10 mM CaCl₂ to improve sealing.

Compounds were evaluated in 8-point concentration-response format (4 replicate wells/concentration). The compound formulations were loaded in e.g. a 384-well compound plate and placed in the Syncropatch plate well.

To monitor the sensitivity the assay, for each plate, the standard antagonist positive control article (Memantine, Tocris) was applied at 8 half log concentrations (range 0.1-300 μM); n=4, where n=the number of replicates per concentration. The agonist positive control (L-glutamate) was applied at eight (8) concentrations (0.03-100 μM; n=4, where n=the number of replicates) together with 50 μM glycine.

2× concentration of test compounds and antagonist control was pre-applied 2minutes before application of L-glutamate/glycine mixed with 1× concentration of test compound. Glutamate and glycine was applied to naive cells (n=4, where n=the number of replicate wells/concentration) via a multi-channel pipettor.

### Electrophysiological Procedures

a) Intracellular solution (mM): 50 mM CsCl, 90 mM CsF, 2 mM MgCl₂, 5 mM EGTA, 10 mM HEPES. Adjust to pH 7.2 with CsOH. This solution was prepared in batches and stored refrigerated. In preparation for a recording session, the intracellular solution was loaded into the intracellular compartment of the multi holes (8×) planar electrode.
b) Extracellular solution, HB-PS (composition in mM): NaCl, 137; KCl, 1.0; CaCl₂, 2; HEPES, 10; Glucose, 10; pH adjusted to 7.4 with NaOH (refrigerated until use).
c) Holding potential: −70 mV, potential during agonist/antagonist application: −70 mV.
Test Compound Administration: The application consisted of the addition of 20 μL of 1× concentrated test article solution and agonist at 40 μL/s (1 second total application time).
Positive Control Agonist: 0.03-100 μM L-glutamate (8 concentration concentration-response, half log scale) and 50 μM glycine, stimulation with 2 μM glutamate, 50 μM glycine.
Positive control antagonist: 0.1-300 μM memantine (8 concentration dose-response, half log scale dilution) co-applied with 2 μM glutamate and 50 μM glycine.

### Recording Procedure

a) Extracellular buffer was loaded into the multi holes plate wells (40 μL per well). Cell suspension was pipetted into the wells (20 μL per well) of the multi holes planar electrode.
b) Whole-cell recording configuration was established via patch perforation with membrane currents recorded by on-board patch clamp amplifiers.
c) Two recording (scans) were performed: First, during test compound and control application alone (2× concentration) and second, during co-application of test compound or antagonist control (1× concentration) with agonist stimulation (˜EC₅₀ L-glutamate) to detect antagonist effects of the compounds.

### Screenpatch Data Analysis

Activation was calculated in two ways based on the following measurements:
1. peak current amplitudes, and
2. current amplitude 4-5 seconds after agonist addition.

Data acquisition was performed via the FLIPR Control software that is supplied with the FLIPR System (MDS-AT) and data was analyzed using Microsoft Excel™ (Microsoft™ Corp., Redmond, WA). Concentration-response data was fitted to a Hill equation of the following form:

$$RESPONSE = Ba se + \frac{Max - Base}{1 + {( \frac{xhalf}{x} )}^{rate}}$$

where Base is the response in the absence of test article (vehicle treatment), Max is the maximum response at high concentrations, xhalf is the EC₅₀, or IC₅₀, the concentration of test compound producing either half-maximal activation or inhibition, and rate is the Hill coefficient. Nonlinear least squares fits were made assuming a simple binding model. If appropriate, fits were weighted by the standard deviation. No assumptions about the fit parameters were made; the fit parameters were determined by algorithm. Raw reduced data was analyzed using Microsoft Excel Office 365 ProPlus (Microsoft™ Corp., Redmond, WA) and XLfit (Excel addon, Copyright© IDBS 2016).

For each plate, a Z′ Factor and Signal Window was calculated in accordance with published online Assay Guidance Manual: https://www.ncbi.nlm.nih.gov/books/NBK83783/#htsvalidation. Bridging_Studies_for_Assay

Preferably, the raw data will meet the following acceptance criteria:
The Z′ factor must be ≥0.4
SW factor must be ≥2.

### Results

Glutamate (plus 50 μM glycine) activated receptors with EC₅₀ of 3.03 μM for PCA and EC₅₀ of 0.82 μM for SSC measurements. All values were calculated relatively to current produced by 100 μM glutamate (E_{MAX} set as 100%, % of control).

The agonist effect of test articles and the positive controls were examined. Table 14 below shows peak current amplitude measurements (PCA) and Table 15—steady state current measurements (SSC). Further data showing any agonist effect can be seen in FIGS. 1 and 3, which show peak current amplitude and steady state current amplitude measurements, respectively.

TABLE 14
Agonist and antagonist effects of test articles on NR1/NR2B
receptors: peak current amplitude measurements.

| | | NR1/NR2B | | | NR1/NR2B | |
|---|---|---|---|---|---|---|
| | | PCA agonist | | | PCA antagonist | |
| | Compound | EMAX, | EC50, | | IC50, | |
| # | ID | % | μM | HillSlope | μM | HillSlope |
| 1 | tartrate | — | — | — | 1.04 | 0.89 |
| 2 | benzoate | — | — | — | 1.18 | 1.39 |
| 3 | Memantine | — | — | — | 5.74 | 1.12 |
| 4 | Glutamate | 100.00 | 3.03 | −0.86 | — | — |
| | CRC | | | | | |

TABLE 15
Agonist and antagonist effects of test articles on NR1/NR2B
receptors: steady state current amplitude measurements

| | | NR1/NR2B | | | NR1/NR2B | |
|---|---|---|---|---|---|---|
| | | PCA agonist | | | PCA antagonist | |
| | Compound | EMAX, | EC50, | | IC50, | |
| # | ID | % | μM | HillSlope | μM | HillSlope |
| 1 | tartrate | — | — | — | 0.31 | 0.79 |
| 2 | benzoate | — | — | — | 0.79 | 1.25 |
| 3 | Memantine | — | — | — | 1.90 | 1.08 |
| 4 | Glutamate | 100.00 | 0.82 | −0.95 | — | — |
| | CRC | | | | | |

Antagonist activity of test articles was examined after stimulation of receptors with 2 μM glutamate and 50 μM glycine in the presence of increasing concentrations of test articles. Two measurements were performed, first was a peak current amplitude and second was a steady state current between 4^{th} and 6^{th} seconds following agonist application. It should be noted that previous studies have shown that open channel blockers (uncompetitive type of inhibition) are more potent at blocking steady state current as compared to peak current amplitude.

Tables 14 and 15 show the calculated IC₅₀ and Hillslope values, while further data showing antagonist effects of the controls and test articles can be seen in FIGS. 2 (PCA measurements) and 4 (SSC measurements).

All controls articles inhibited NR1/NR2B NMDA receptors having an IC₅₀ in the range of 1.04-1.17 μM for PCA measurements and 0.31-0.49 μM range for SSC measurements. SSC was inhibited to a somewhat greater extent.

Reference negative allosteric modulator, ifenprodil, inhibited NR1/NR2B NMDA receptors with an IC₅₀ of 1.67 μM and 0.54 μM for PCA and SSC measurements respectively.

Reference antagonist, memantine, inhibited NR1/NR2B NMDA receptors with an IC₅₀ of 5.74 μM and 1.90 μM for PCA and SSC measurements respectively.

### Conclusion

In conclusion, ifenprodil benzoate inhibits glutamate and glycine activation of NR1/NR2B receptors in vitro at a level comparable to ifenprodil tartrate and to the control antagonists when PCA and SSC measurements were taken.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

We claim:

1. A compound comprising an ifenprodil salt selected from the group consisting of ifenprodil oleate, ifenprodil nicotinate, ifenprodil pamoate, ifenprodil fumarate, and ifenprodil benzoate.

2. A method of treating or preventing a disorder or condition comprising administering an ifenprodil salt selected from the group consisting of ifenprodil oleate, ifenprodil nicotinate, ifenprodil pamoate, ifenprodil fumarate, and ifenprodil benzoate to a subject in need thereof.

3. Use of an ifendprodil salt selected from the group consisting of ifenprodil oleate, ifenprodil nicotinate, ifenprodil pamoate, ifenprodil fumarate, and ifenprodil benzoate for the treatment or prevention of a disorder or condition in a subject in need thereof.

4. A compound comprising an ifenprodil salt selected from the group consisting of ifenprodil oleate, ifenprodil nicotinate, ifenprodil pamoate, ifenprodil fumarate, and ifenprodil benzoate for the treatment or prevention of a disorder or condition in a subject in need thereof.

5. The method, use, or compound of any one of claims 1 to 4, wherein the disorder or condition is mood affective disorders; neurotic, stress-related and somatoform disorders including anxiety disorders; disorders of psychological development; behavioral syndromes associated with physiological disturbances and physical factors; extrapyramidal and movement disorders; episodic and paroxysmal disorders, epilepsy; pain; forms of neurodegeneration; cerebrovascular diseases, acute and chronic; and any sequelae of cerebrovascular diseases.

6. The method, use, or compound of any one of claims 1 to 4, wherein the disorder or condition is bipolar disorder, major depressive disorder, treatment-resistant depression, schizophrenia, ante- and postpartum depression, seasonal affective disorder, Alzheimer's disease, dementia, Parkinson's disease, Huntington's chorea, multiple sclerosis, cognitive impairment, head injury, spinal cord injury, stroke, epilepsy, movement disorders including dyskinesia, neurodegenerative diseases including amyotrophic lateral sclerosis and neurodegeneration associated with bacterial or chronic infections, glaucoma, pain including chronic, cancer, post-operative and neuropathic pain, diabetic neuropathy, migraine, cerebral ischemia, encephalitis, autism and autism spectrum disorders, memory and learning disorders, obsessive compulsive disorder, attention deficit hyperactivity disorder (ADHD), PTSD, tinnitus, sleep disorders including narcolepsy and excessive daytime sleepiness, vertigo and nystagmus, anxiety autoimmunological disorders including neuropsychiatric systemic lupus erythematosus, addictive illnesses including alcohol addiction and drug addiction, viral infections, acute lung injury, liver disease related neurological alterations, Korsakoff's disease, cerebral palsy, neuronal reperfusion injury, neuronal hemorrhage, tinnitus, neuronal exposure to a toxic substance, elevated cholesterol, idiopathic pulmonary fibrosis, fibrotic injury, radiation induced fibrosis, and/or chronic cough.

7. The method, use, or compound of any one of claims 1 to 6, further comprising administration with a compound that modulates NR2A-containing NMDA receptors.
